SEQUENCE LISTING (2 sequences)

SEQ ID NO: 1 - DNA, 30 bp, Artificial Sequence
  Description of Artificial Sequence Synthetic primer
gtacggatcctatatcttggccctttggaa

SEQ ID NO: 2 - DNA, 30 bp, Artificial Sequence
  Description of Artificial Sequence Synthetic primer
gtcactcgagcagtactttccaaatgcctc

(19) United States
(12) Patent Application Publication
(10) Pub. No.: US 2012/0053208 A1
(43) Pub. Date: Mar. 1, 2012

(54) Curcumin Analogs as Dual JAK2/STAT3 Inhibitors and Methods of Making and Using the Same

(75) Inventors: Pui-Kai Li, Galloway, OH (US); Chenglong Li, Dublin, OH (US); Jiayuh Lin, Dublin, OH (US); James R. Fuchs, Columbus, OH (US)

(73) Assignee: THE OHIO STATE UNIVERSITY RESEARCH FOUNDATION, Columbus, OH (US)

(21) Appl. No.: 13/264,580

(22) PCT Filed: Apr. 15, 2010
(86) PCT No.: PCT/US2010/031207

§ 371 Date: Nov. 10, 2011

Related U.S. Application Data
(60) Provisional application No. 61/169,440, filed on Apr. 15, 2009.

(51) Int. Cl. A61K 31/4465; C07D 309/06; C07D 211/32; A61K 31/12; C12Q 1/48; A61P 35/00; A61P 3/10; A61P 29/00; C12Q 1/02; C12Q 1/68; C07C 49/577; A61K 31/351

(52) U.S. Cl. 514/330; 568/329; 568/330; 549/425; 546/225; 514/683; 514/684; 514/451; 435/29; 435/6.1; 435/15

(57) ABSTRACT

Curcumin analogues and methods of making and using the same are disclosed.

### CROSS-REFERENCE TO RELATED APPLICATIONS

This application claims the benefit of U.S. Provisional Application Ser. No. 61/169,440 filed Apr. 15, 2009, the entire disclosure of which is expressly incorporated herein by reference.

### STATEMENT REGARDING FEDERALLY SPONSORED RESEARCH

This invention was not made with Government support Grant. No. R21CA133652-01 awarded by the National Institutes of Health. The government has certain rights in the invention.

### SEQUENCE LISTING

The instant application contains a Sequence Listing which has been submitted via EFS-web and is hereby incorporated by reference in its entirety. The ASCII copy, created on Apr. 12, 2010, is named 604_{—}50803_SEQ_LIST_{—}09002.txt, and is 777 bytes in size.

### TECHNICAL FIELD AND INDUSTRIAL APPLICABILITY OF THE INVENTION

The present invention relates to compositions and methods for detecting, treating, characterizing, and diagnosing cancer-related diseases. More particularly, the present invention provides curcumin analogues and methods of making and using the same.

### BACKGROUND OF THE INVENTION

Curcumin, 1,7-bis(4-hydroxy-3-methoxyphenyl)-1,6-heptadien-3,5-dione, is the primary bioactive compound isolated from turmeric, the dietary spice made from the rhizome of Curcuma longa. Turmeric has been a mainstay of traditional Indian folk medicine, and it has been used for the treatment of many diseases such as diabetes, liver disease, rheumatoid arthritis, atherosclerosis, infectious diseases and cancers. The therapeutic effects of curcumin are attributed to its activity on a wide range of molecular targets. One of the most important aspects of curcumin is its effectiveness against various types of cancer with both chemopreventive and chemotherapeutic properties. While curcumin is reported to show little to no toxicity (no dose-limiting toxicity at doses up to about 10 g/day in humans), the utility of curcumin is limited due to poor bioavailability and poor selectivity. The lack of selectivity is due to the numerous molecular targets with which curcumin is known to interact. These include several targets closely associated with cancer cell proliferation such as the STAT transcription factors.

Therefore, it would be useful to have effective compositions that are more effective than curcumin in inhibiting the JAK/STAT pathway

It would also be useful to have methods of treating different types of cancer-related disorders, such as solid tumors, and hematopoietic cancers using such compositions.

### SUMMARY OF THE INVENTION

In a first aspect, there are provided herein curcumin analogues, as schematically illustrated in the Figures herein. Non-limiting examples include dialkylated dimethoxycurcumin analogues, curcumin analogues having an aromatic substituent; curcumin analogues having a benzaldehyde aromatic substituent; curcumin analogues having a having a mono-, di-, and tri-substituted benzaldehyde substituent containing methoxy (and hydroxy) groups.

In another aspect, there are provided herein methods for synthesizing curcumin analogues, as schematically illustrated in the Figures herein.

In another aspect, there are provided herein pharmaceutical compositions at least one curcumin analogue as described herein.

In another aspect, there are provided herein methods of treating a cancer-related disease comprising modulating the activity of a one or more of JAK and STAT in a subject in need thereof, by administering at least one curcumin analogue described herein.

In another aspect, there are described herein methods for inhibiting JAK/STAT signaling in a subject in need thereof, comprising administering one or more of the curcumin analogues described herein.

In another aspect, there is provided herein an intermediate systemic in vivo xenograft system comprising: implanting cancer cells just under a chicken embryo chorioallantoic membrane (CAM) away from major vessels; treating the CAM with a tolerated dose of a composition being tested by administering in an area distal from the implantation location; after a period of time, excising around the area of implantation, and imaging the excised CAMs.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIG. 1—Prior Art: Inhibitors of JAK/STAT pathway. 1—Peptide and peptidomimetic STAT 2 SH2 dimerization inhibitors. 2—Small molecule STAT3 SH2 dimerization inhibitors. 3—JAK2 inhibitors. 4—Inhibitors derived from natural products.

FIG. 2a: Structures of curcumin analogues labeled “FLLL31” and “FLLL32.”

FIG. 2: Structures of additional curcumin analogues.

FIG. 3: FLLL31 and FLLL 32 inhibit STAT3 phosphorylation and induce apoptosis in MDA-MB-231 breast cancer cells. Cells were treated with 2.5 and 5 μM of FLLL31 and FLLL32 for 24 h. The cell extract were processed for immunoblotting using specific antibodies. GAPDH was used as loading control.

FIG. 4: FLLL31 and FLLL32 inhibit STAT3 phosphorylation and induce apoptosis in MDA-MB-468 breast cancer cells. Cells were treated with 2.5, 5 and 10 μM of curcumin analogs for 24 h. The cell extracts were processed for immunoblotting using specific antibodies. GAPDH was used as loading control.

FIG. 5: FLLL31 (10 μM), FLLL32 (10 μM) and curcumin (10 μM) inhibit STAT3 DNA binding activity in MDA-MB-231 breast cancer cells. Cells were treated with the compounds or DMSO (control) for 24 h. The cell extract were analyzed for STAT3 binding activity using STAT3 Transcription Factor Kit.

FIG. 6: FLLL31 and FLLL32 inhibit STAT3 but not STAT1 DNA binding activity in MDA-MB 231 breast cancer cells. Cells were treated with FLLL31 (10 μM) or DSMO (control) for 24 h. The cell extracts were analyzed for STAT3 DNA binding activity using STAT3 and STAT1 Transcription Factor Kits.

FIGS. 7a-11b: FLLL31 (FIG. 7a) and FLLL32 (FIG. 7b) show a dose dependent inhibition of STAT3-depenendt transcription luciferase activity in MDA-MB231 breast cancer cells. Cells were incubated with the compounds for 24 h and were then harvested for luciferase activity analysis.

FIG. 8: FLLL32 (10 μM) inhibited the stimulation of STAT3 phosphorylation by IL-6 and INF-α in MDA-MB-453 breast cancer cells. FLLL32 did not inhibit the stimulation of STAT1 and STAT2 phosphorylation in INF-α in MDA-MB-453 cells. Cells were pre-incubated with FLLL32 for 2 h and then treated with IL-6 and INF-α for 0.5 h and collected.

FIG. 9: FLLL32 inhibit STAT3 phosphorylation and induce apoptosis in BXPC-3 pancreatic cancer cells. Cells were treated with 5 and 10 μM of FLLL32 for 24 h. The cell extracts were processed for immunoblotting using specific antibodies. GAPDH was used as loading control.

FIG. 10: FLLL32 inhibit STAT3 phosphorylation and induce apoptosis in U266 multiple myeloma cells. Cells were treated with 2.5, 5 or 10 μM of FLLL32 and or 5 μM of curcumin for 24 h. The cell extracts were processed for immunoblotting using specific antibodies. GAPDH was used as loading control.

FIG. 11: FLLL32 and WP1066 inhibit STAT3 phosphorylation and induce apoptosis in U373 human glioblastoma cells. Cells were treated with 5 μM of FLLL32 or WP1006 for 24 h. The cell extracts were processed for immunoblotting using specific antibodies. GAPDH was used as loading control.

FIG. 12: JAK2 inhibitory activities of FLLL31, FLLL32, AG490, WP1066 and curcumin. JAK2 kinase assay was performed using a HTScan JAK2 kinase assay kit. Statistical significance (P<0.05) relative to the DSMO is designated by an (*).

FIG. 13a: Table 1—IC₅₀ (μM) of FLLL31 and FLLL32 and other JAK2.5TAT3 or STAT3 dimerization inhibitors in human breast cancer (B), pancreatic cancer (P), glioblastoma (G), and multiple myeloma (MM) cells expressing activated STAT3.

FIG. 13b: FLLL31, FLLL32 (5 or 10 μM) induce apoptosis in PANC-1, BXPC-3 and SK-BR-cells with persistent expression of p-STAT3, but no apoptosis induction in non-malignant human pancreatic duct epithelial cells (HPDE), normal human mammary epithelial cells (HMEC), and normal human lung fibroblasts (W-38). Cells were treated with various concentrations of FLLL31 or FLLL32 for 24 h. The cell extracts were processed for immunoblotting using specific antibodies. GAPDH was used as loading control.

FIG. 14: Molecular docking model of diketone tautomer of curcumin with JAK2 (left) and STAT3 protein (right).

FIGS. 15a-15c: Effect of FLLL32 on vascularity and tumor growth in CAMs. Human MDA-MB-231 metastatic breast cancer cells were implanted in the CAMs of chicken embryos and drugs given at the dosed indicated at days 1 and 3 post tumor implantation and tumors imaged at day 4 post-implantation. FIG. 15a-Blood vessel density around xenografted tumors (“t”). FIG. 15b-Relative blood vessel density. FIG. 15c-Relative tumor sized of xenografts. Data are from 7-8 individual embryos and 2 separate experiments. *P<0.05, **P<0.01, ***<P 0.001 by one-way ANOVA with Neuman-Keuls post-test.

FIG. 16: Pilot pharmacokinetic data for curcumin and FLLL32. Mice were dosed IP (50 mg/kg) or IV (25 mg/kg) with curcumin or FLLL32 (12.5 mg/ml in DMSO). Blood was collected from individual mice at each time point between 2 min. and 4 hours, and plasma concentrations of parent compounds was measured via LCMSMS. Determined concentrations below the linear range (i.e., below 10 nM) are not displayed.

FIG. 17: Fluorescant polarization of fluorescent p-Tyr peptide as a function of STAT3 concentrations.

FIGS. 18a-18b: Computational model of the two tautomeric forms of curcumin (FIG. 18a—diketone form; FIG. 18b—enol form) binding to JAK2.

FIG. 19a: Table 2—Predicted binding energy for curcumin and analogues with JAK2 and STAT3.

FIG. 19b: Series 1 analogues and Series 2 analogues of dialkylated dimethoxycurcumin analogues.

FIG. 19c: Table 3—Effect of phenol substitution of predicted binding energy.

FIG. 19d: Table 4—Affect of various alkyl groups on the central bond angles.

FIG. 19e: Scheme 1—Synthesis of Series 2 analogues.

FIG. 19f: Scheme 2—Synthesis of Series 1 analogues.

FIG. 19g: Table 5—Antiproliferative activity of monoketone curcumin derivatives against breast cancer cells (MDF-7 and MDA-MB231). MCF-10A cells (breast epithelial cells) are used as a “normal” tissue model.

FIG. 20: Examples of aromatic substituents for dialkylated curcumin analogues.

FIG. 21: Examples of benzaldehydes for the synthesis of analogues.

FIG. 22: Examples of mono-, di-, and tri-substituted benzaldehydes containing only methoxy (and hydroxy) groups useful for the synthesis of analogues.

FIG. 23a: Examples of non-symmetric JAK2 inhibitors 24 and 25.

FIG. 23b: Scheme 3—Synthesis of non-symmetric analogues as JAK2 inhibitors.

FIG. 23c: Scheme 4—Acylation of methyl ketone 26a.

FIG. 24: The key binding pockets, or “hot-spots” for the STAT3 SH2 domain.

FIG. 25a: Structure of the cyclohexyl derivative 6a of curcumin.

FIG. 25b: Docking of 6a in the STAT3 SH2 binding site.

FIG. 26a: FLLL32 and analogues. Log P values are calculated using ChemDraw Ultra 11.0.

FIG. 26b: Scheme 5—Synthetic scheme for the synthesis of compounds 32-25.

FIG. 27a: Analogues targeting the Leu706 site of STAT3.

FIG. 27b: Scheme 6—Preparation of compound 36.

FIG. 28: Sulfamate and phosphate analogues of FLLL31 (R=Me) and FLLL32 (R-cyclohexyl).

FIG. 29a: Sulfamate and phosphate compounds.

FIG. 29b: Scheme 7—Synthesis of model system for synthesis of sulfamate and phosphate derivatives.

FIG. 29c: Scheme 8—Synthesis of sulfamate and phosphate analogues of FLLL31.

FIG. 29d: Scheme 9—Synthesis of the FLLL32 derivative containing one free phenol.

FIG. 29e: Examples of analogues for compound 58.

FIG. 29f: Examples of analogues for compound 59.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

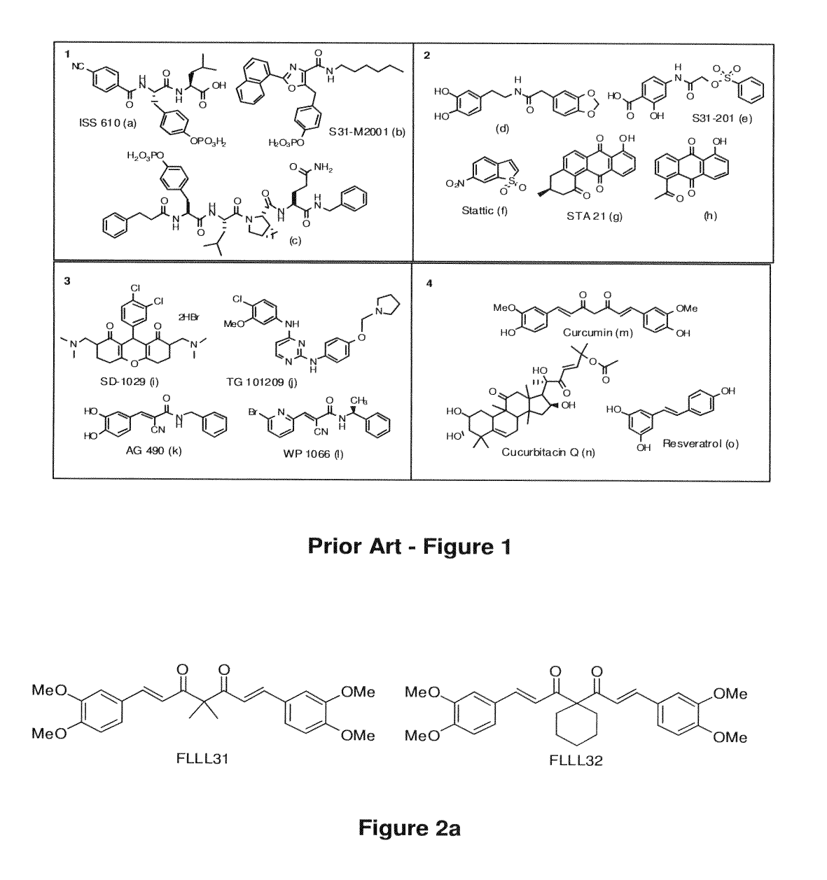

Several small molecule dimerization inhibitors are shown in Prior Art—FIG. 1. One inhibitors includes cucurbitacin Q, which inhibits STAT3 phosphorylation, although the actual biochemical target(s) is still unclear, and the indirubin derivative, E804, which inhibits both Src and STAT3. The catechol containing synthetic molecule AG-490 is a selective JAK2 inhibitor, but it suffers from poor in vivo stability. Several other JAK2 inhibitors include WP1066, SD-1008, SD-1029 and TG101348. Among all of the polyphenols, curcumin is the most widely studied compound in chemoprevention and chemotherapy.

Curcumin and Tumorigenesis

Curcumin, 1,7-bis(4-hydroxy-3-methoxyphenyl)-1,6-heptadien-3,5-dione (Prior Art—FIG. 1), is the primary bioactive compound isolated from turmeric, the dietary spice made from the rhizome of Curcuma longa. Turmeric has been a mainstay of traditional Indian folk medicine and it has been used for the treatment of many diseases such as diabetes, liver disease, rheumatoid arthritis, atherosclerosis, infectious diseases and cancers. The therapeutic effects of curcumin are attributed to its activity on wide range of molecular targets. One of the most important aspects of curcumin is its effectiveness against various types of cancer with both chemopreventive and chemotherapeutic properties. Unlike most chemotherapeutic agents, curcumin shows little to no toxicity (no dose-limiting toxicity at doses up to 10 g/day in humans). Unfortunately, the potential utility of curcumin is somewhat limited due to poor bioavailability and poor selectivity. The lack of selectivity is due to the numerous molecular targets with which curcumin is known to interact.

There is evidence that the therapeutic activity of curcumin is partially through inhibition of the JAK/STAT pathway. Curcumin has been shown to inhibit JAK2, Src, Erb2, and EGFR, all of which are implicated in STAT3 activation. Furthermore, curcumin has been shown to downregulate the expression of Bcl-xL, cyclin D1, VEGF, and TNF all of which are known to be regulated by STAT3. There is also evidence which implicates a number of important STAT3 target genes in the formation of tumors.

The present invention is based, at least in part, on the inventors' discovery that the impact of the central the diketone moiety on structure and biological activity is more significant than that of the aromatic substituents. The inventors herein have also discovered that inhibition of JAK/STAT signaling by curcumin plays a significant role in its chemotherapeutic and chemopreventive properties.

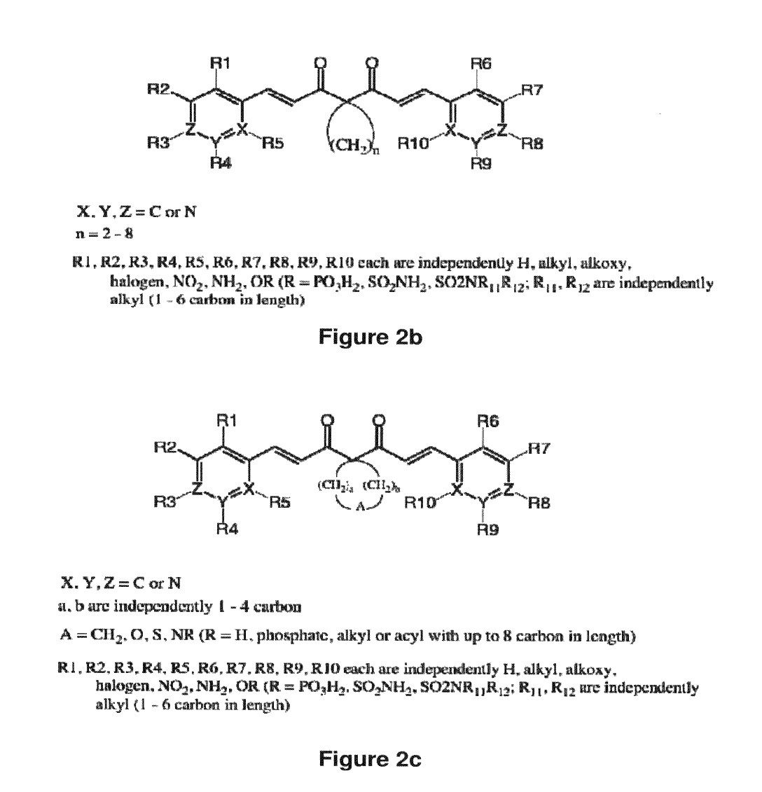

The inventors have designed and synthesized two diketone analogues of curcumin (FLLL31 and FLLL32). The analogues labeled as “FLLL31” and “FLLL32” (FIG. 2a) and additional analogues shown in FIG. 2b and FIG. 2c are specific inhibitors of the JAK2/STAT3 pathway.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified. The value of the present invention can thus be seen by reference to the Examples herein.

### Example I

### FLLL31 and FLLL32 Inhibit STAT3-Phosphorylation in Breast Cancer Cells with Constitutively Active STAT3

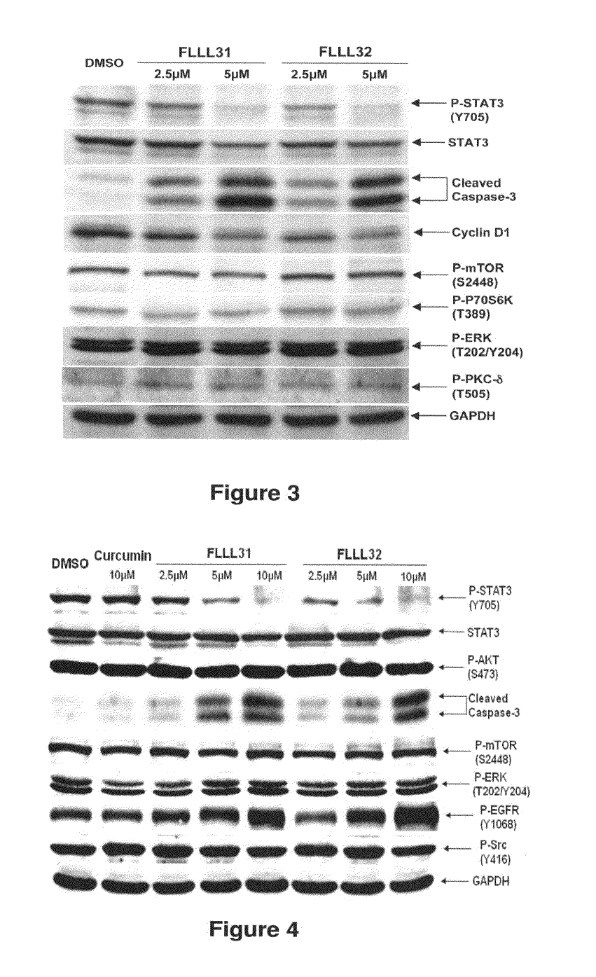

The inventors examined whether FLLL31 and FLLL32 inhibited STAT3 phosphorylation in MDA-MB-231 and MDA-MB-468 breast cancer cells, which expressed persistently tyrosine phosphorylated [at tyrosine residue 705 (Y705)] or activated STAT3. FLLL31 and FLLL32 inhibited STAT3 phosphorylation in MDA-MB-231 (FIG. 3) and MDA-MB-468 (FIG. 4) human breast cancer cell lines. FLLL31 and FLLL32 have little effects on ERK1/2, PKC-δ, mTOR, p70S6K, and AKT phosphorylation (FIG. 3 and FIG. 4).

The inhibition of STAT3 phosphorylation by FLLL31 and FLLL32 is consistent with the induction of apoptosis evidenced by the cleavages of caspase-3. Further, FLLL31 and FLLL32 cause the down-regulation of cyclin D1, a downstream target of STAT3 in both breast cancer cell lines (FIG. 3 and FIG. 4).

Inhibition of STAT3 DNA Binding and STAT3-Dependent Luciferase Activities by FLLL31 and FLLL32.

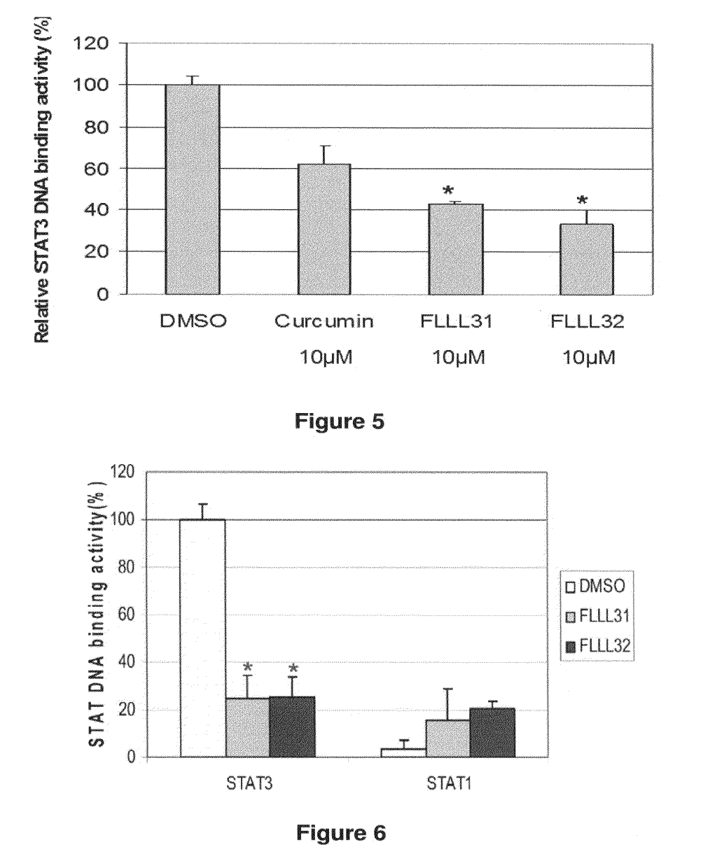

To confirm the inhibition of STAT3 signaling by FLLL31 and FLLL32, the inventors examined the abilities of the compounds to inhibit STAT3 DNA binding and STAT3-dependent transcriptional luciferase activities. Both FLLL31 and FLLL32 caused a statistically significant inhibition of STAT3 DNA binding activity in MDA-MB-231 cells and were significantly more potent than curcumin (FIG. 5).

Furthermore, both FLLL31 and FLLL32 showed selectivity to inhibit STAT3 but not STAT1 DNA binding activity (FIG. 6).

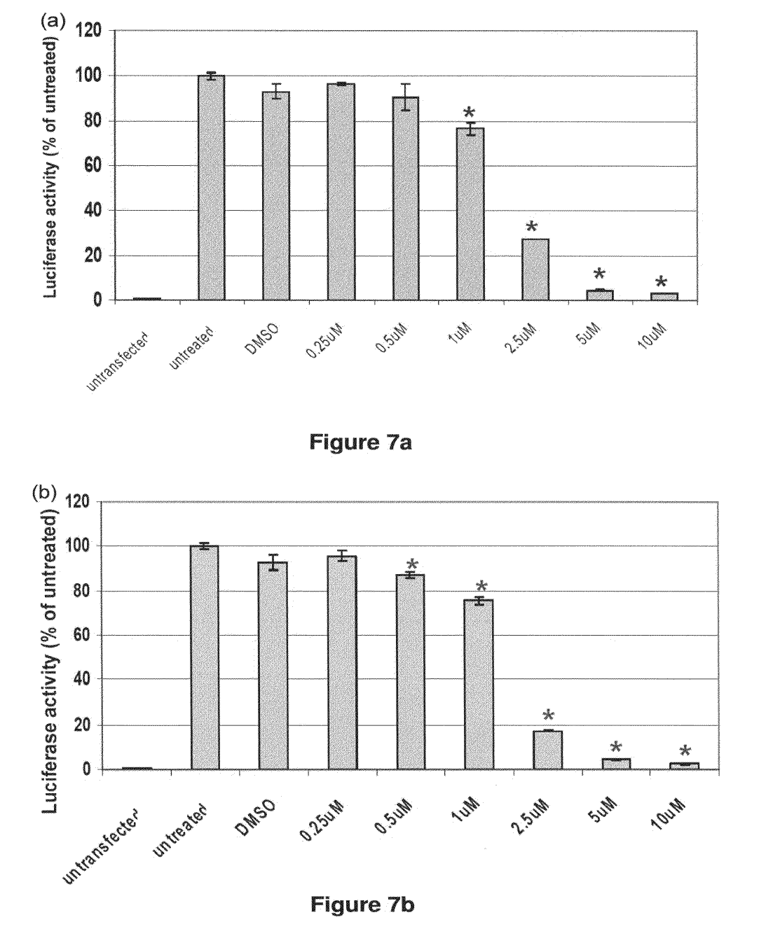

Due to its high endogenous levels of phosphorylated or activated STAT3 protein, MDA-MB-231 breast cancer cells were chosen to be stably transfected with pLucTKS3, a luciferase construct that features seven copies of the STAT3 binding site in a thymidine kinase minimal reporter. Expression of luciferase is thus contingent upon the phosphorylation and activation of STAT3. These stably transfected cells were treated with 1-10 μmol/L of FLLL31 and FLLL32 for 24 hours. Luciferase activity was monitored via a luminometer, and the luminescence of the FLLL31 and FLLL32-treated cells were compared to that of an untreated control. Following normalization of the data, both FLLL31 and FLLL32 were shown to cause a dose-dependent inhibition of STAT3 dependent luciferase activity (FIG. 7).

FLLL32 Inhibits the Stimulation of STAT3 Phosphorylation by IL-6 but does not Inhibit the Stimulation of STAT1 and STAT2 Phosphorylation by IFN-α

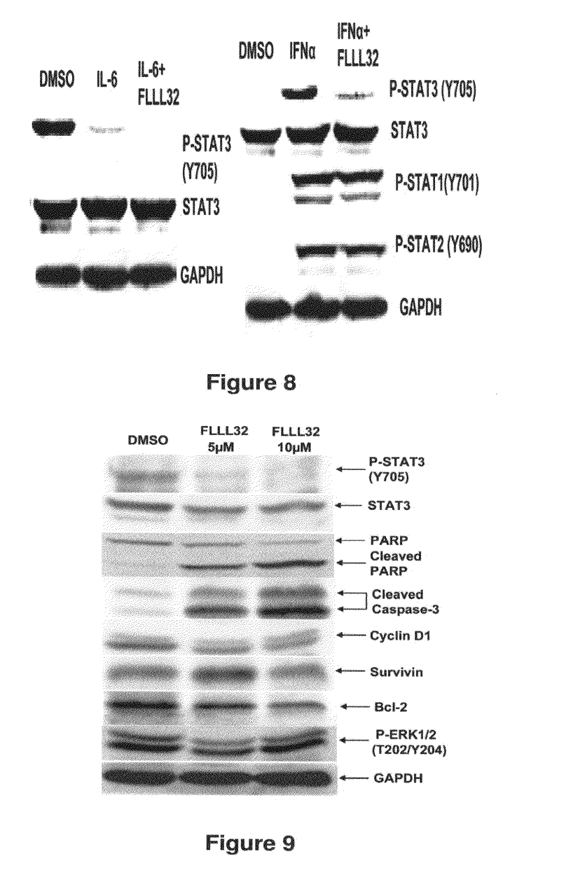

Since IL-6 induces STAT3 phosphorylation and may play a role in cancer development, the inventors examined whether FLLL32 inhibits this induction. IL-6 stimulates STAT3 phosphorylation and is inhibited by FLLL-32 (FIG. 8). It was also observed that IFN-α induces STAT3 phosphorylation and is inhibited by FLLL32. However, FLLL32 does not inhibit the induction of STAT1 and STAT2 phosphorylation by IFN-α (FIG. 8). This indirectly indicated that FLLL32 does not inhibit JAK1 or TYK2.

Inhibition of STAT3 Phosphorylation in Non-Breast Cancer Cell Lines by FLLL32

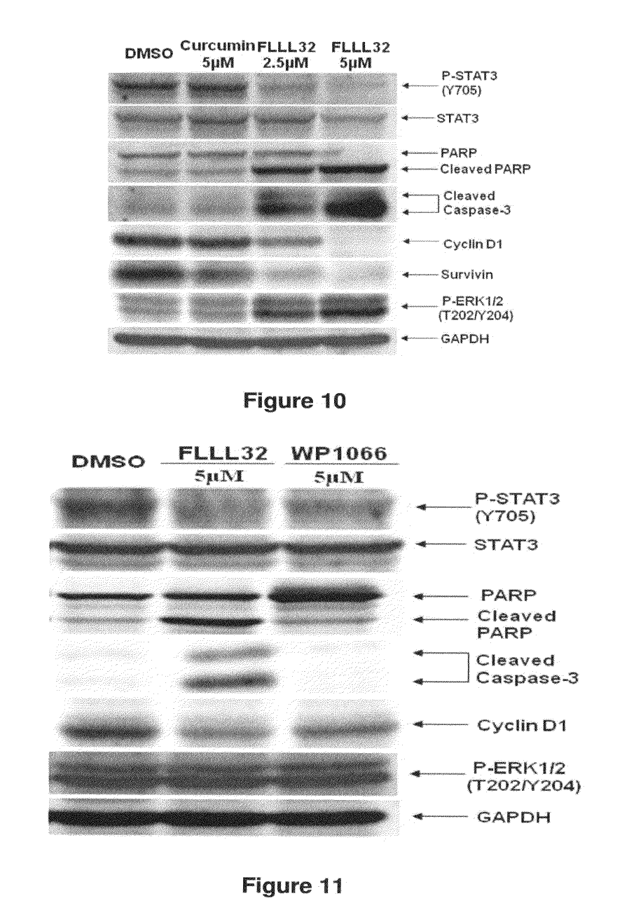

We next examined whether FLLL32 also inhibited STAT3 phosphorylation in non-breast cancer cell lines BXPC-3 (human pancreatic cancer, FIG. 9), U266 (multiple myeloma, FIG. 10) and U373 (human glioblastoma, FIG. 11). All three cell lines express persistently activated STAT3. FLLL32 inhibited STAT3 phosphorylation and induced apoptosis in all three cell lines (FIGS. 9-11). It also down-regulated the downstream targets of STAT3 such as ERK1/2, cyclin D1, Bcl-2, and survivin (FIGS. 9-11).

Inhibitory Activities of FLLL31, FLLL32, Curcumin, AG490 and WP-1066 on JAK2 Kinase.

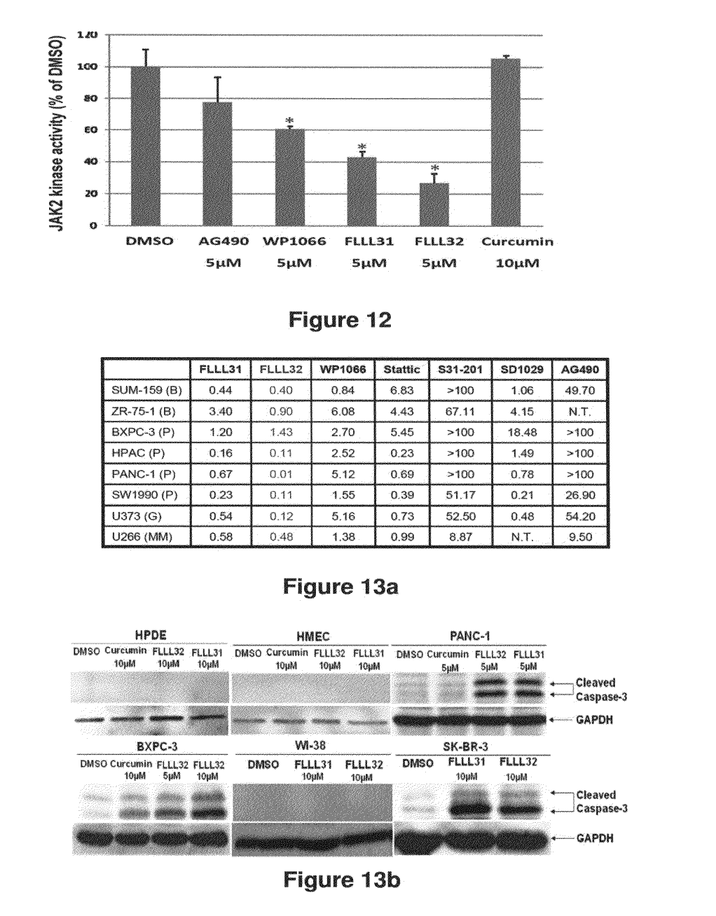

JAK2 mediates the phosphorylation of STAT3 at tyrosine residue 705 in response to cytokine signaling. Therefore, we examined whether FLLL32 directly inhibits JAK2 kinase activity. FLLL32 is more potent than FLLL31 and curcumin to inhibit JAK2 kinase activity (FIG. 12). FLLL32 is also more potent than two other known JAK2 inhibitors, WP1066 and AG490 (FIG. 12). At 10 μM, curcumin did not show any JAK2 inhibition. While not wishing to be bound by theory, the inventors now believe that these results explain the inhibition of STAT3 phosphorylation by FLLL32.

Selective Cytotoxicity of FLLL32 on Cancer Cells with Constitutively Active STAT3 Over Normal Cells.

The inventors further examined whether FLLL32 would also induce apoptosis in normal human cells without expressing persistent STAT3 phosphorylation. FLLL32 did not induce detectable apoptosis in normal human pancreatic duct epithelial cells, normal human lung fibroblasts, or normal human mammary epithelial cells. It did, however, induce apoptosis (as evidenced by cleaved caspase-3) in PANC-1 and BXPC-3 pancreatic cancer cells and SK-BR-3 breast cancer cells (FIG. 13a).

Antiproliferative Activities of FLLL31, FLLL32 and Other Known Inhibitors of the JAK/STAT Pathway.

The inventors also examined the anti-proliferative activities of both FLLL31,

FLLL32 and compared them with several known inhibitors (WP1066, Stattic, S31-201, SD1029 and AG490) of the JAK/STAT pathway against a panel of eight cancer cell lines with elevated levels of STAT3 phosphorylation including breast (SUM-159, ZR-75-1), pancreatic (BXPC-3, HPAC, PANC-1, SW1990), glioblastoma (U373) and multiple myeloma (U266). Both FLLL31 and FLLL32 are more potent than the other inhibitors with IC₅₀ values in submicromolar concentrations. FLLL32 appears to be slightly more potent than FLLL31 (FIG. 13b—Table 1).

Molecular Docking Study of the Diketone Tautomer of Curcumin with the JAK2 (ATP Binding Site) and STAT3 (SH2 Domain).

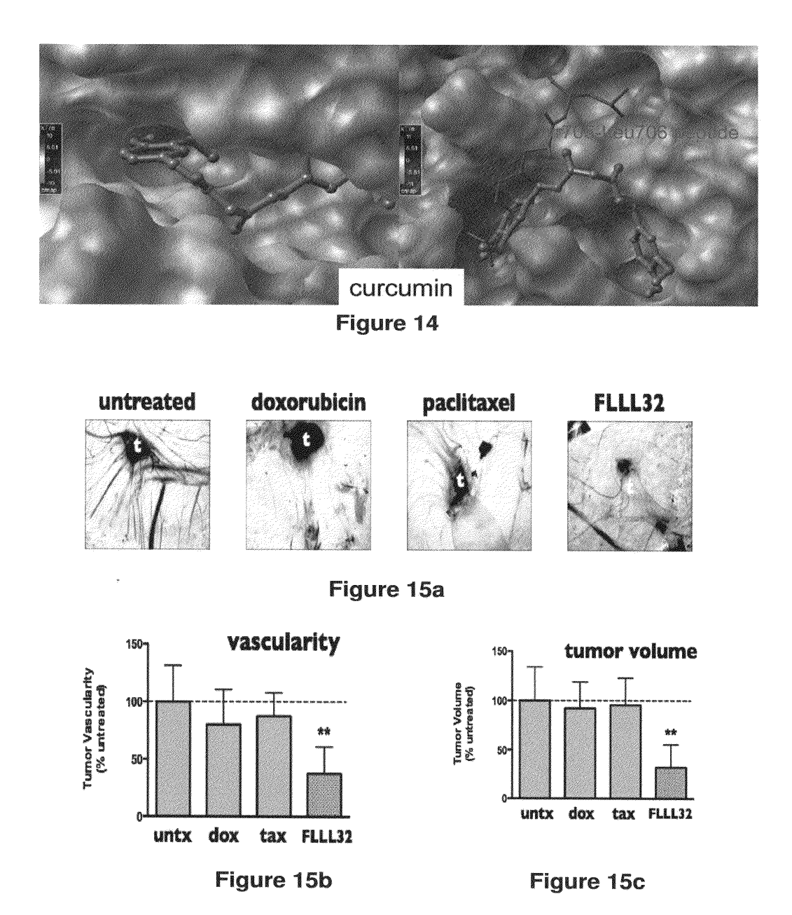

A molecular docking study was carried out to examine the role of the 1,3-diketone moiety of curcumin in the binding to JAK2 and STAT3. FIGS. 14a-14b show the bent binding modes of diketo-curcumin. For JAK2 binding (FIG. 14a—left), the aromatic fragments compete with the purine ring of ATP on the left side and bind to a largely hydrophobic pocket on the other. In addition, one of the carbonyl oxygens in the center of the molecule interacts with the JAK2 oxyanion hole. With regard to STAT3 SH2 dimerization site binding, the aromatic fragments similarly compete with the pTyr705 binding site on the left side and bind to a largely hydrophobic side pocket on the right (FIG. 14b—right). The red stick shows the pTyr705-Leu706 peptide binding mode from the other SH2 domain during STAT3 dimerization and activation.

Effect of FLLL32 on Vascularity and Tumor Growth in Chicken Chorioallantoic Membrane (CAM) Xenograft Assay.

The inventors developed an intermediate systemic in vivo xenograft system using the chicken embryo chorioallantoic membrane (CAM). Specifically, MDA-MB-231 human metastatic breast tumor cells (250,000), shown to overexpress STAT3 were implanted just under the CAM of 10 day of incubation (DI) chicken embryos away from major vessels in 50 μL of an inert human extracellular matrix (Humatrix). One day after implantation, embryos were treated with 80% of the maximum tolerated dose (MTD)—(based on 11 DI weight) of FLLL32 (25 mg/kg), or doxorubicin (2 mg/kg) or paclitaxel (2 mg/kg) systemically by pipetting onto the CAM in an area distal from the implantation location. Three days after implantation, CAMs were fixed in situ using 0.1% triton X-100 in 4% paraformaldehyde for 2 minutes, excised around the area of implantation, fixed and spread into 6-well plates containing 4% paraformaldehyde. These excised CAMs were then imaged on a brightfield dissecting microscope at 6.25× magnification (Wild M400 Photomakroscop).

It was found that 25 mg/kg FLLL32 reduced the number of intact blood vessels surrounding implanted tumors (“t” in FIG. 15a) whereas neither doxorubicin nor paclitaxel treatment resulted in any significant change in tumor vascular density (FIG. 15a and FIG. 15b).

Further, FLLL32 resulted in significant reduction in MDA-MB-231 tumor volume whereas doxorubicin or paclitaxel had no effect (FIG. 15c).

Taken together, these data indicate that FLLL32 has a significant anti-tumor and anti-angiogenic effect on STAT3 overexpressing breast cancer CAM xenografts.

FLLL32 Pharmacokinetics in ICR Mice.

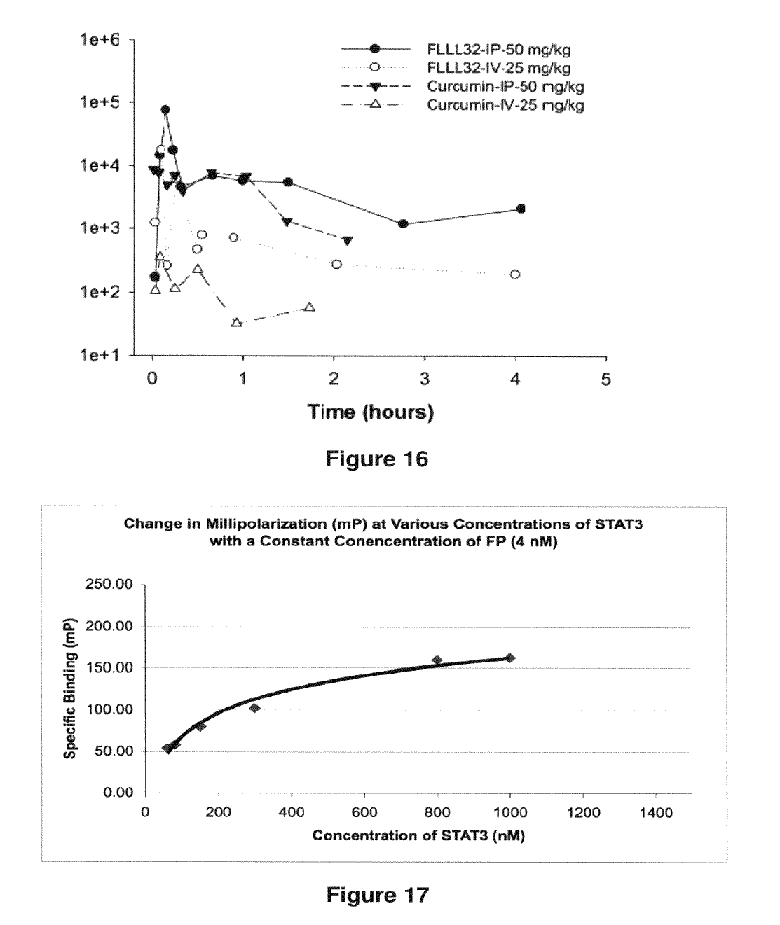

To estimate the overall PK time course for future definitive PK/PD studies, a pilot study was conducted in ICR mice administered both IP and IV doses of FLLL32 and curcumin. Male ICR mice, 8-10 weeks, were dosed IP (50 mg/kg) or IV (25 mg/kg) with either FLLL32 or curcumin in DMSO (12.5 mg/mL). Mice were exsanguinated under isoflurane anesthesia via cardiac puncture at various times between 2 min. and 4 hrs, and plasma was recovered from collected blood samples and stored at −70° C. until analysis. Tissues from a subset of mice were collected and stored for later work to develop efficient extraction procedures of curcumin derivatives from tissue. Curcumin and FLLL32 were quantified via LCMSMS analysis. Extracted plasma samples (100 μL) were dried under vacuum then reconstituted in 80% acetonitrile containing 0.1% formic acid. Injected samples (20 μL) were separated through a C-18 column (50×2.1 mm, 3 μm) with constant 0.4 mL/min flow with a gradient of water and acetonitrile, each containing 0.1% formic acid. Eluted analytes were detected via single reaction monitoring on a Quantum TSQ Discovery Max using atmospheric pressure chemical ionization in positive mode. A curcumin analogue was used as an internal standard, and analyte/IS ratios enabled quantitation via a standard curve produced in mouse plasma. The linear range used for this assay was 10 nM to 1 μM for both compounds, and samples measuring above 1 μM were diluted for repeat analysis. Resulting concentration vs. time profiles for each compound and dosing route are shown in FIG. 16.

Again, while not wishing to be bound by theory, the inventors herein now believe that FLLL32 may have increased exposure and potentially longer half-life compared to curcumin, as indicated by the lower AUCs and more rapid disappearance of curcumin (i.e., curcumin concentrations fell below the 10 nM cutoff for quantitation).

Fluorescent Polarization (FP) Assay Development and Optimization.

The molecular docking studies showed that FLLL32 could bind to the STAT3 SH2 domain.

In addition, the inventors herein used a fluorescent polarization assay to determine whether FLLL32 and/or its analog compounds would bind to the SH2 domain. The development of fluorescence polarization was established. Briefly, the assay was performed in black 384-well microplates (Perkin Elmer, Waltham, Mass.) in total volume of 25 μL in each well. The fluorescence intensity values were recorded using excitation filter at 540 nm and emission filter at 590 nm. FP measurements were executed by setting the integration time of 100 ms, an excitation filter at 545 nm and emission filter at 610 nm.

All data is expressed in millipolarization unit. The mP values were calculated using the equation mP=1000×[(I II−I⊥)/(I II+I⊥)]I II: parallel emission intensity measurement, I⊥: perpendicular emission intensity measurement. Saturation curves were recorded in which fluorescently labeled peptide (4 nM) was treated with increasing amounts of recombinant STAT3 protein. The specific binding was defined as the contribution to signal of bound peptide and was calculated as mP=mPb−mPf, where mPb and mPf are the polarization value values of bound and free tracer, respectively; mP is the recorder polarization value for a specific STAT3 concentration. The calculated dissociation constant (Kd) is 172 nM (FIG. 17), which is in alignment with binding affinities between pTyr peptides and Stat SH2 domains.[83].

### Example II

### Evaluation of Curcumin Analogues Based on FLLL32 as JAK2 and STAT3 Inhibitors

Curcumin presents an excellent lead compound for the development of novel anticancer agents. The highly modular structure and relative ease of synthesis facilitates both the rapid and systematic preparation and evaluation of a highly varied library of analogous compounds to explore the structure-activity relationship of this molecule with regard to JAK2 and STAT3 activity.

The Example II focuses on the preparation and evaluation of derivatives featuring the same structural motif found in FLLL32, a dialkylated curcumin analogue shown to be a potent inhibitor of the JAK/STAT pathway (see Example I).

The derivatives of curcumin can be optimized for JAK2 and STAT3 activity independently to obtain greater potency and specificity toward these targets. While not wishing to be bound by theory, the inventors herein now believe that even relatively minor structural modifications designed to improve activity against a single protein target may ultimately result in a decrease in activity with respect to another.

1.1 Optimization of Curcumin Analogues for JAK2 Activity.

The curcumin scaffold was modified to show the effects of structural changes on JAK2 activity. This approach involves: 1) the synthesis of a series of 4,4-dialkylated curcumin derivatives which enforce the diketone tautomeric form and interact with pocket 2 of the JAK2 binding site, 2) variation of the aromatic ring substituents to improve potency and selectivity through binding to the phosphotyrosine pocket of JAK2, and 3) the synthesis of non-symmetric derivatives in order to assess the importance of the second aromatic ring (and its substituents) in JAK2 binding.

1.1.1. Synthesis of 4,4-Disubstituted Curcumin Derivatives.

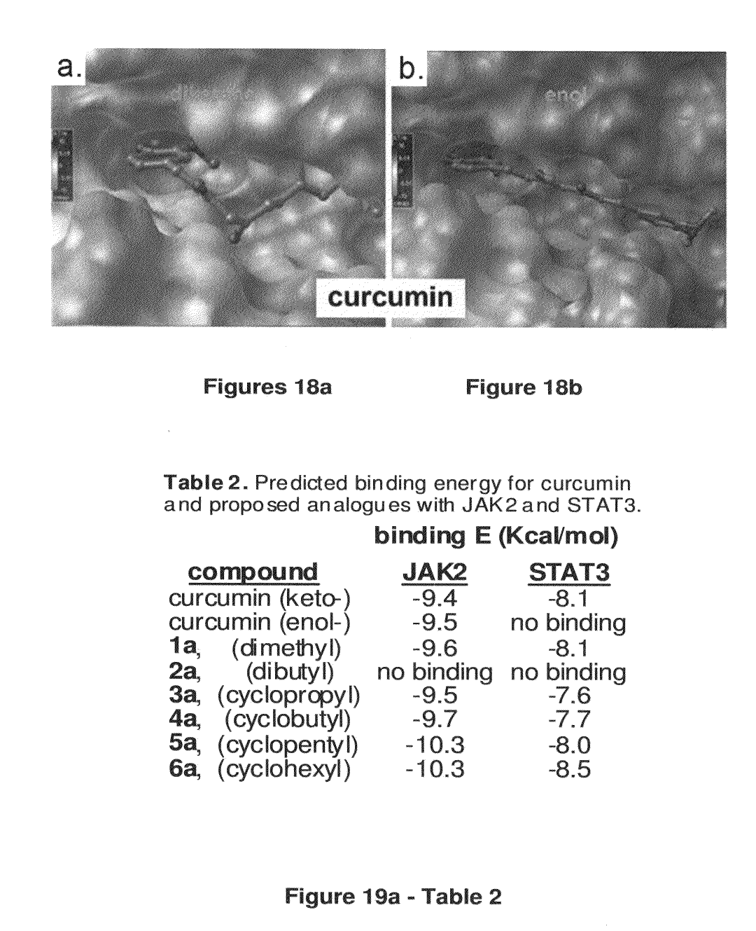

As discussed in the Example I, a molecular docking study was carried out to examine the role of the 1,3-diketone moiety of curcumin in the binding to JAK2 (and STAT3). The keto and enol forms of curcumin were both employed. Interestingly, the two tautomeric forms displayed similar predicted binding energies. FIG. 18a shows the bent binding mode of diketo-curcumin with JAK2. The key binding interactions are derived from competition of one aromatic fragment with the purine ring of ATP on the left side and the binding of the other aromatic ring to a largely hydrophobic pocket (pocket 2) on the other. In addition, one of the carbonyl oxygens in the center of the molecule interacts with the JAK2 oxyanion hole.

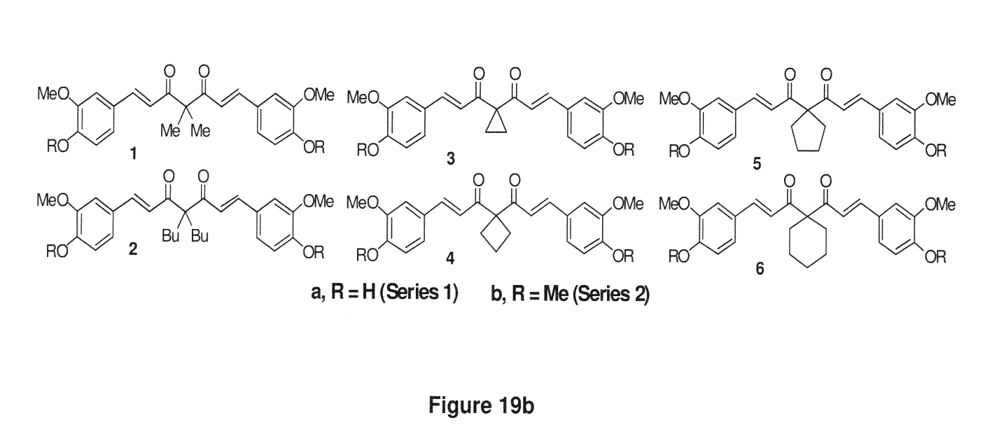

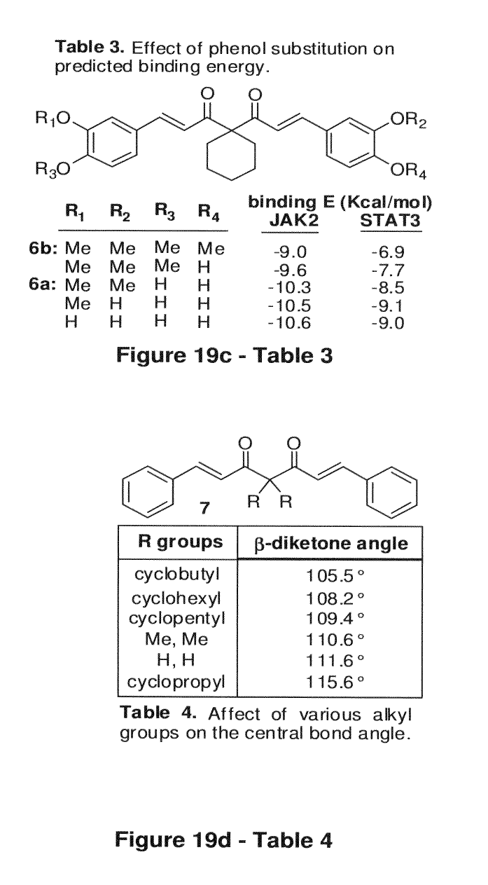

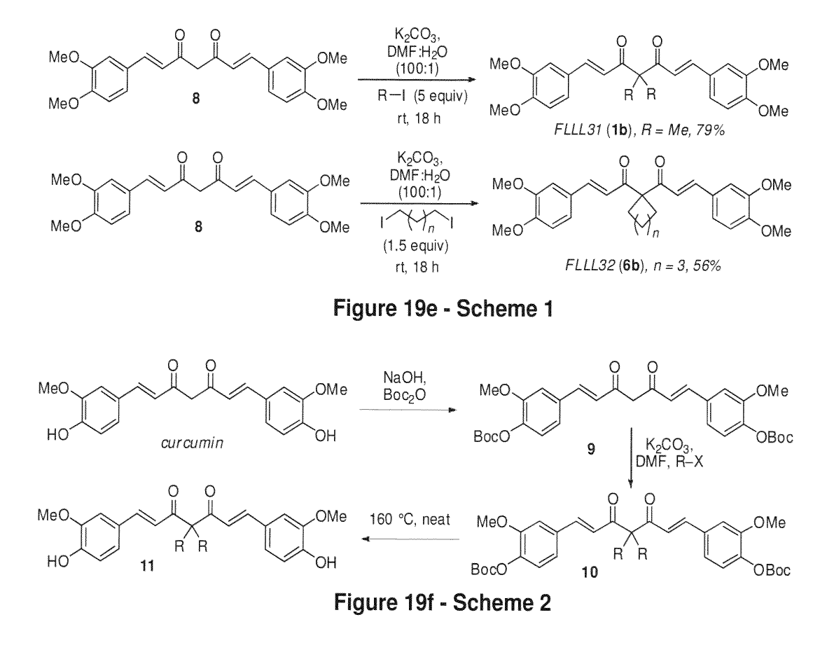

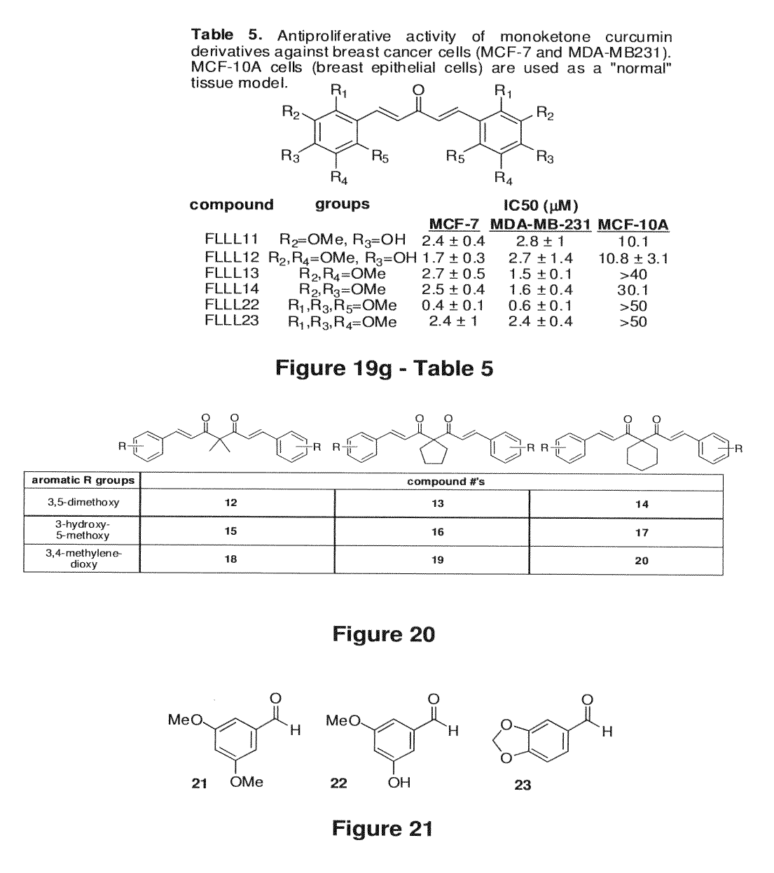

Based on the results of this initial docking study, a second computational study was executed to examine the conformational and steric effects of disubstitution at the C-4 position of curcumin (FIG. 19a—Table 2 and FIG. 19b).

This series of symmetrical analogues differs from curcumin only by the presence of the two central alkyl substituents (Series 1). This substitution effectively locks the compounds into the diketone tautomeric form, eliminating the possibility of enolization. In our docking studies, the spiro-cyclopentyl and -cyclohexyl derivatives, 5 and 6 (FIG. 19b), show the best binding affinity at the molecular level to JAK2 (FIG. 19a—Table 2). Thus, although the enol tautomer of curcumin is predicted to bind well to JAK2, derivatives of the keto tautomer will be pursued due to the predicted improvement in binding energy displayed by compounds 5 and 6.

In both of these cases, in addition to the purine-competing aromatic binding and carbonyl oxyanion hole interaction, the hydrophobic alkyl rings (cyclopentyl- and cyclohexyl-) are believed by the inventors herein to interact favorably with pocket 2 of JAK2. This is evidence that the diketone tautomer of curcumin is important for JAK/STAT activity and that careful alterations to this scaffold can lead to potent and selective JAK2 inhibitors.

Another series of compounds with additional methyl substituents on the phenolic oxygens is shown (Series 2, FIG. 19b). The inventors' computational model shows that hydrogen bonding interactions of the free phenols found in series 1 are important for binding to both JAK2 and STAT3 (FIG. 19c—Table 3). However, the increased potency observed for various curcumin analogues lacking free phenols, specifically dimethoxycurcumin (8, FIG. 19e—Scheme 1), may be due to increased stability and increased levels of concentration in plasma. Thus, the hydroxyl substituents at the 4-position of the aromatic rings may contribute significantly to observed solubility and stability issues of these curcumin-like compounds, especially at neutral to basic pH. Therefore, Series 2, containing alkylated derivatives of dimethoxycurcumin which have no free phenols, can be prepared, such as, for example, Compound 6b.

Although the binding energies of these compounds are predicted to be slightly lower due to a lesser degree of hydrogen bonding (i.e., 6b vs. 6a, FIG. 19c—Table 3), these compounds are predicted to be more stable and membrane permeable, perhaps leading to an increase in in vivo activity.

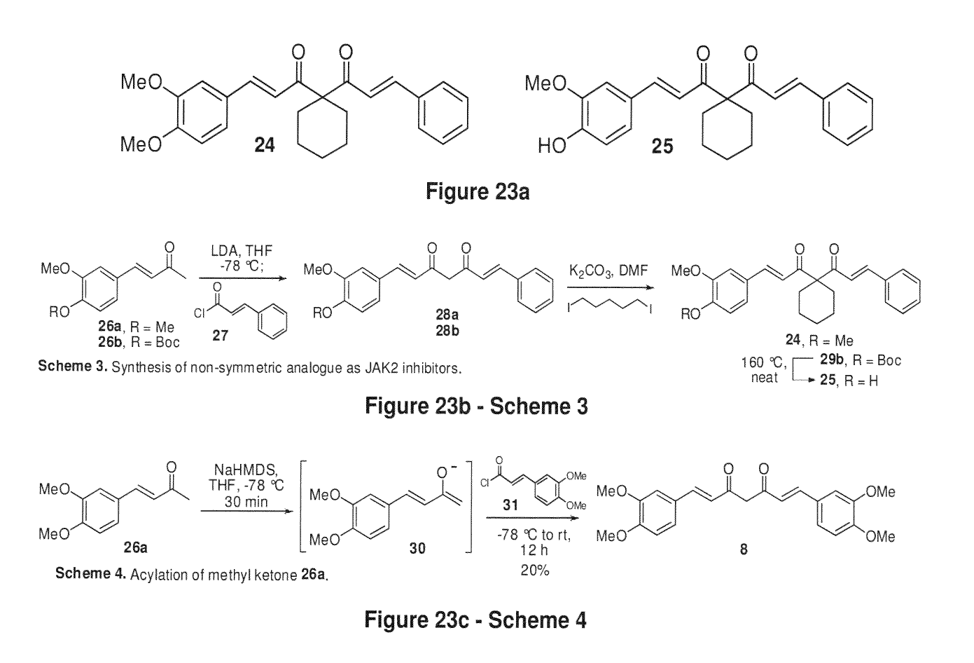

The synthesis of the two series of curcumin analogues is useful to probe the nature of pocket 2 and validate the inventors' binding model using small alkyl substituents (dimethyl 1, cyclopropyl 3) and sterically bulkier, but more lipophilic, alkyl substituents (di-n-butyl 2, cyclohexyl 6). The various alkyl substituents have a measurable affect on molecular conformation since the angle between the two carbonyl groups varies dramatically (from 105.5° to 115.6°) due to the nature of these groups (FIG. 19d—Table 4). The synthesis of these compounds of Series 2 can be carried out according to FIG. 23e—Scheme 1.

Dimethoxycurcumin (8) will be prepared via condensation of 3,4-dimethoxybenzaldehyde and 2,4-pentanedione according to the procedure of Venkateswarlu. Treatment of 8 with potassium carbonate in the presence of a suitable alkylating agent is expected to affect the desired disubstitution reactions. Alkylation with the diiodoalkanes should result in formation of the spirocyclic products.

As noted in Example I, the inventors now prepared derivatives FLLL31 (1b, R=methyl) and FLLL32 (6b, cyclohexyl) in this way. Interestingly, O-alkylation of the enolate generated from 8 is also observed in both cases, although the yield of this product is relatively low (<10%) and can be readily separated via column chromatography.

The synthesis of members of Series 1, containing 4-hydroxy, 3-methoxy substituted aromatic rings, is challenging, requiring the use of a suitable protecting group in order to affect the desired alkylation reaction. The inventors have now, however, established a synthetic route applicable to these compounds employing a t-butyloxycarbonyl (Boc) protecting group strategy (FIG. 19—Scheme 2). In this synthetic route, curcumin, prepared using condensation conditions, can be utilized as the starting material. Protection of curcumin using t-butyldicarbonate provides the bis-protected curcumin derivative 9. Alkylation of this derivative using potassium carbonate as base affects the disubstitution reaction in analogy to our preliminary results. Finally, removal of the Boc protecting groups via thermolysis furnishes the desired analogues of general structure 11.

For example, this procedure has been successfully applied to the synthesis of the corresponding cyclohexyl derivative 6a (FIG. 19b). Initial attempts to protect/deprotect the phenols using other protecting groups, including benzyl ether or silyl ether groups, have failed to provide the desired products. Similarly, attempts to remove the Boc protecting groups under standard protic acid conditions also led to significant decomposition of starting material with no observed product.

1.1.2. Variation of the Aromatic Substituents of Dialkylated Curcumin Analogues.

The inventors herein now believe that the computational model of curcumin bound to JAK2 shows that the substituents on the aromatic ring in the phosphotyrosine pocket play a critical role in binding potency. While not wishing to be bound by theory, the inventors herein now believe that their analysis of the pocket indicates that hydrogen bonding interactions (both hydrogen bond donor and hydrogen bond acceptor interactions) may be key to this binding potency.

In addition, the inventors' research on related curcumin derivatives containing only a single carbonyl moiety indicates that, although derivatives with varied aromatic substituents frequently display similar antiproliferative activity toward cancer cells, the effect on “healthy” model cells (e.g., MCF-10A) can vary dramatically (FIG. 19g—Table 5).

Thus, modification of the aromatic ring substituents beyond those shown in Section 1.1.1 herein (3-methoxy-4-hydroxy and 3,4-dimethoxy) can be used to examine: 1) the role of the substituents in JAK2 activity will be probed; and 2) selectivity of the drugs against cancer cells in order to reduce toxicity.

An additional series of analogues (FIG. 20), derived from the commercially available or readily prepared benzaldehydes 3,5-dimethoxybenzaldehyde (21), 3-hydroxy-5-methoxybenzaldehyde (22), and piperonal (3,4-methylenedioxybenzaldehyde, 23) (FIG. 21), can be employed to further determine the variations in activity caused by different ring substitution patterns. These benzaldehydes have been selected to make only minor perturbations to the steric and electronic environments of the aromatic systems present in the previous section. Compounds 12-17 can participate in the critical hydrogen bonding interactions with the proteins; further the change of the substituent location on the ring from the C-4 to the C-5 position can increase stability to both basic and oxidative conditions.

In certain embodiments, this is particularly important since the cleavage of curcumin to the reactive catechol and subsequent oxidation to the ortho-quinone is thought to be an operative metabolic pathway. The variation of the methoxy (12-14) and hydroxyl substituents (15-17) should also provide more information on the nature and significance of the hydrogen bonding in the JAK2 pocket. The piperonal derived compounds 18-20, which have the methylated phenols tied back into a less sterically demanding and slightly less hydrophobic acetal, are designed to directly mimic the 3,4-dimethoxy substituted compounds.

For the preparation of these analogues, the alkylation reactions will likewise be carried out on substrates analogous to 8 in FIG. 19e—Scheme 1. No protecting groups will be necessary for the 3,5-dimethoxybenzaldehyde or piperonal derived compounds. The 3-hydroxy-5-methoxybenzaldehyde, however, will need to be protected as the Boc derivative. The substituents on the central carbon of the curcumin scaffold in this example will be limited to dimethyl, cyclopentyl, and cyclohexyl groups. The cyclopentyl and cyclohexyl substituted compounds can be made according to this scheme. Also, the docking studies indicate that these compounds have the best binding interactions with pocket 2 (FIG. 19a—Table 2). For example, Compounds such as—1a, 4a, 5a and 6a have been made.

In addition, the dimethyl substituted derivatives will also be prepared to examine the effects of acyclic substituents. The inventors herein also believe that that the results of Section 1.1.1 may show another dialkyl group which demonstrates more effective JAK2 or antiproliferative activity against cancer cells, and such substituents are also within the contemplated scope of the present invention.

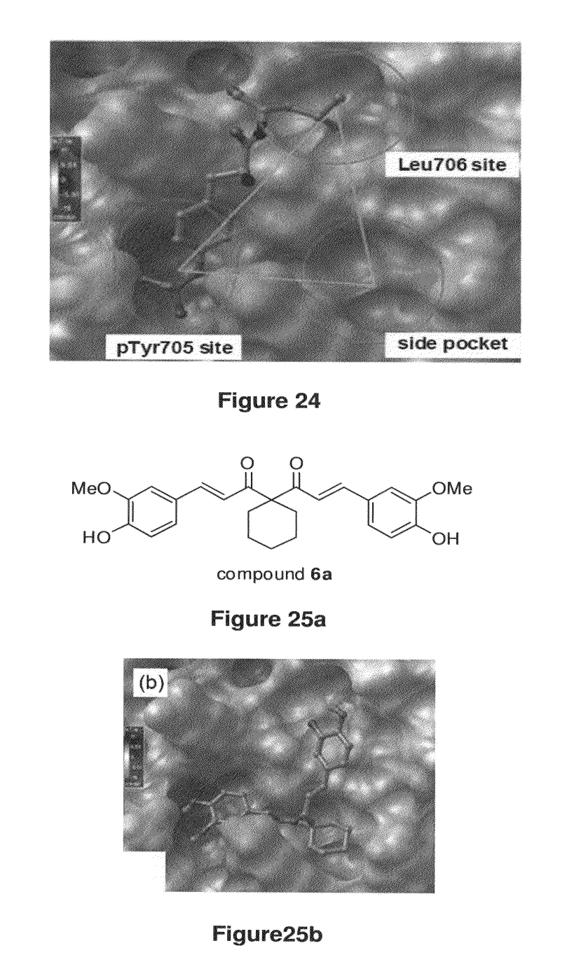

Computational study can also be carried out in order to identify potential ring substituents with more favorable interactions in the JAK2 phosphotyrosine pocket, leading to increased potency. As illustrated in FIG. 24, the number of mono-, di-, and trisubstituted benzaldehydes containing only methoxy and hydroxy groups is quite high.

Rather than synthesizing all of the possible combinations of curcumin analogues based on these aldehydes, computational chemistry allowed the inventors to examine a focused library of compounds and to determine which derivatives can be prepared.

This approach will also be expanded to other structurally varied, but commercially available or readily synthesized benzaldehydes and heteroaromatic compounds. Hits derived from this in silico screening will then be synthesized according to the same synthetic strategy described herein

1.1.3. Synthesis of Non-Symmetric Analogues as Potential JAK2 Inhibitors.

The two aromatic rings of curcumin are predicted to reach into both the phosphotyrosine and hinge link regions of JAK2, respectively. Hydrogen bonding interactions in both of these binding pockets may, or may not, be critical for activity. The binding ability of the curcumin derivatives prepared in Examples 1.1.1 and 1.1.2 may actually be positively influenced by the symmetric nature of the scaffold.

This symmetry allows the analogues to effectively hydrogen bond within these pockets regardless of the relative orientation of the molecule (i.e., both aromatic rings bind equally well). In order to test this hypothesis, two key analogues (24 and 25) will be synthesized which lack substitution on one of the aromatic rings of the curcumin scaffold (the right side of the molecules in FIG. 23).

The synthesis of these derivatives was executed starting with either methyl ketone 26a or 26b. These methyl ketones are available via Wittig olefination of the corresponding benzaldehydes. Formation of the enolate of the methyl ketone upon treatment with base and subsequent acylation using acid chloride 27 provided the curcumin derivative 28a or 28b. Subsequent alkylation of these products provided the desired compound 24 or the Boc protected derivative 29b, respectively. Heating of 29b resulted in the removal of the Boc group to provide 25.

The inventors herein have recently examined the feasibility of this acylation strategy via reaction of ketone 26a with acid chloride 31 (FIG. 23c—Scheme 4). The product obtained in this example, dimethoxycurcumin (8), has been synthesized by the inventors. Upon purification, the product of the reaction was confirmed to be identical to the previously prepared material. Further optimization of these reaction conditions can be carried out in order to increase the yield of the reaction.

In certain embodiments, should application of this acylation reaction strategy to the preparation of the desired compounds prove difficult, however, alternative synthetic routes can also be employed. For example, simultaneous condensation of 2,4-pentanedione with both a 3,4-disubstituted benzaldehyde and benzaldehyde itself can provide a mixture of curcumin derivatives. Chromatographic separation of these products can provide the desired curcumin derivative 28a or 28b along with the two corresponding symmetric curcumin derivatives.

In certain embodiments, however, a more efficient alternative can be the application of a stepwise condensation of the benzaldehydes with 2,4-pentanedione.

1.2 Optimization of Curcumin Analogues for STAT3 Activity.

The curcumin scaffold can also be modified to determine the effect of structural changes on STAT3 activity. As indicated in Example I, a computational study of the binding of curcumin to STAT3 was initiated in addition to the JAK2 study (Section 1.1.1).

Contrary to the results of the JAK2 binding study, however, only the diketone form of curcumin, which is able to adopt a “bent” conformation in the STAT3 binding site, was predicted to bind efficiently. This result led the inventors to identify three key “hotspots” in the STAT3 SH2 domain which may provide increased potency and selectivity: the pTyr705 site, a hydrophobic side pocket, and the Leu706 site (FIG. 24).

The pTyr705 site is quite similar to the phosphotyrosine pocket of JAK2, indicating that structural modifications designed to target the analogous JAK2 pocket may also be applicable to STAT3 binding. In addition to the screening of the compounds prepared in Example 1.1 for STAT3 activity, two additional synthetic strategies can also be employed to increase potency based on our computational model of the STAT3 SH2 domain: 1) variation of the size and lipophilicity of the cyclohexyl moiety predicted to bind to the hydrophobic pocket of the SH2 domain and 2) the synthesis of non-symmetric analogues of FLLL32 designed to target the Leu706 binding pocket.

1.2.1. Variation of the Central Cyclohexane Moiety.

FLLL32 (6b) is a STAT3 inhibitor; the computational model of the closely related 6a bound to the STAT3 SH2 domain demonstrates the key interactions for this class of compounds

As illustrated in FIGS. 25a-25b, the left aromatic ring (pTyr705 site), right aromatic ring (Leu706 site), and the cyclohexyl ring (hydrophobic pocket) are now believed by the inventors herein to bind to all three “hotspots” of the SH2 domain, resulting in increased STAT3 activity and selectivity.

Despite the key role of the central cyclohexyl ring, however, the highly-hydrophobic nature of this particular group may negatively impact the effectiveness of the molecule in vivo by affecting its solubility properties. Therefore, in order to improve upon the solubility of FLLL32, a small number of analogous derivatives containing spirocyclic rings can also be prepared.

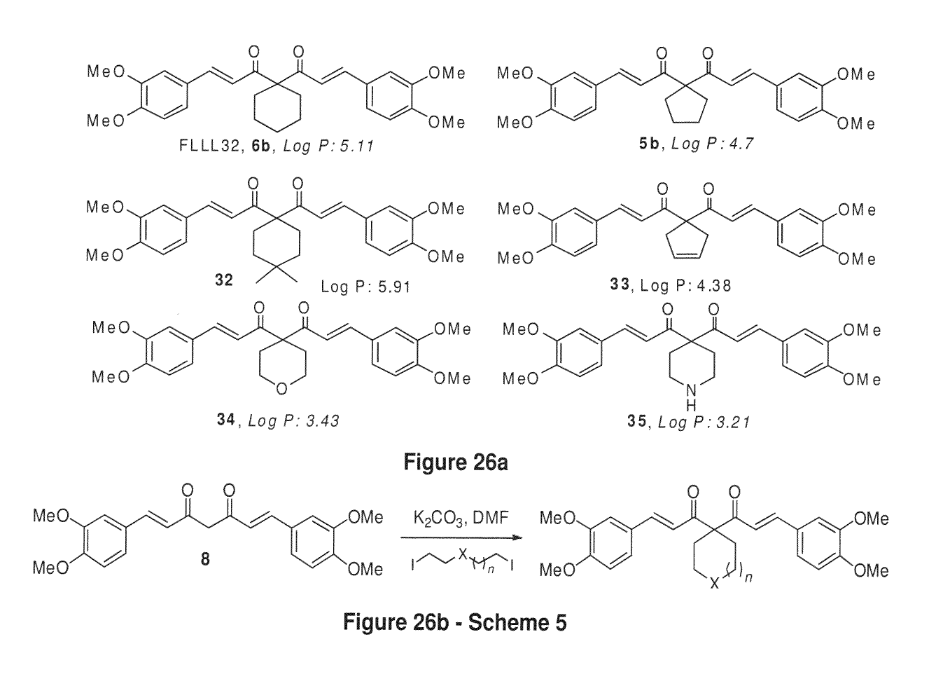

The analogues (FIG. 26a) are designed to compliment the series of dialkylated analogues (see FIG. 19, Example 1.1.1) through variation of the size (5b, 32, and 33) and hydrophobicity (34 and 35) of the central spirocyclic ring. The cyclopentane derivative 5b is described in Example Aim 1.1.1. However, the inventors also note that in this case a small structural change (six-membered ring to five-membered ring) has a fairly significant impact on the predicted logP value. The inventors now believe that it binds to STAT3 with nearly the same binding energy as FLLL32.

The cyclopentene derivative 33 is slightly less hydrophobic, although sterically it should also be able to occupy the hydrophobic binding pocket of STAT3.

Finally, compound 32 containing geminal dimethyl substituents can be synthesized to determine the overall size of the pocket. Introduction of heteroatoms in compounds 34 and 35 can increase the water solubility more significantly.

Compound 35 is also an attractive compound because the piperidine nitrogen may ultimately be functionalized to selectively target cancer cells. The synthesis of these compounds can be accomplished using substitution reactions shown in FIG. 26b-Scheme 5). The commercially available iodoethylether necessary for the alkylation of dimethoxycurcumin (8) can be purchased while the remaining alkylating agents are synthesizable according to established procedures.

1.2.2. Targeting the Leu706 site: Non-Symmetric Analogues of FLLL32.

Further improvement in binding potency and selectivity can be achieved by targeting the Leu706 site of STAT3. The introduction of relatively short alkyl chains (ethyl<propyl<iso-butyl) at the 3-position of one aromatic ring can have a profound effect on the potency and selectivity of these molecules for STAT3.

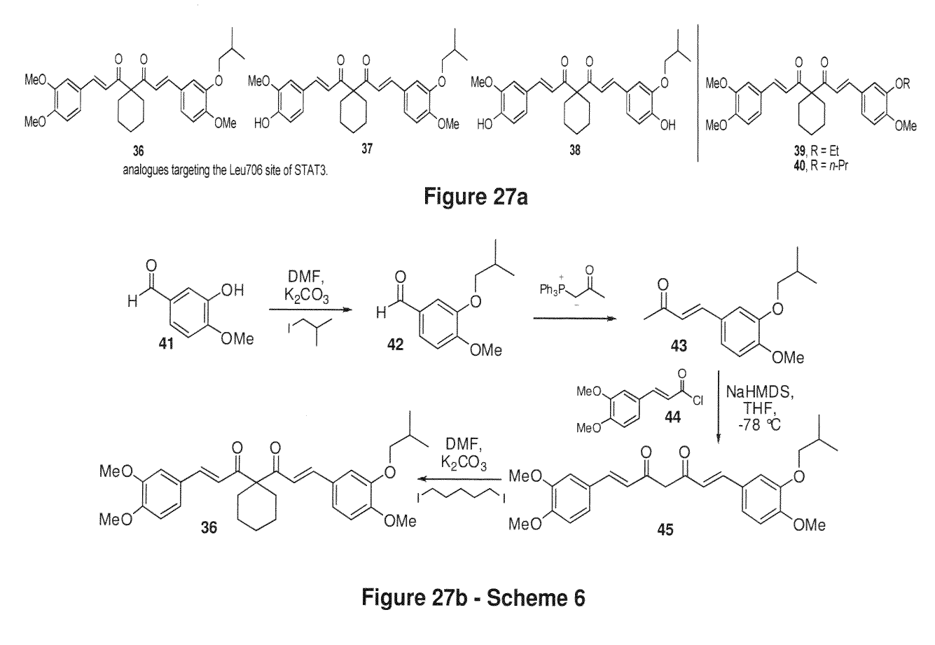

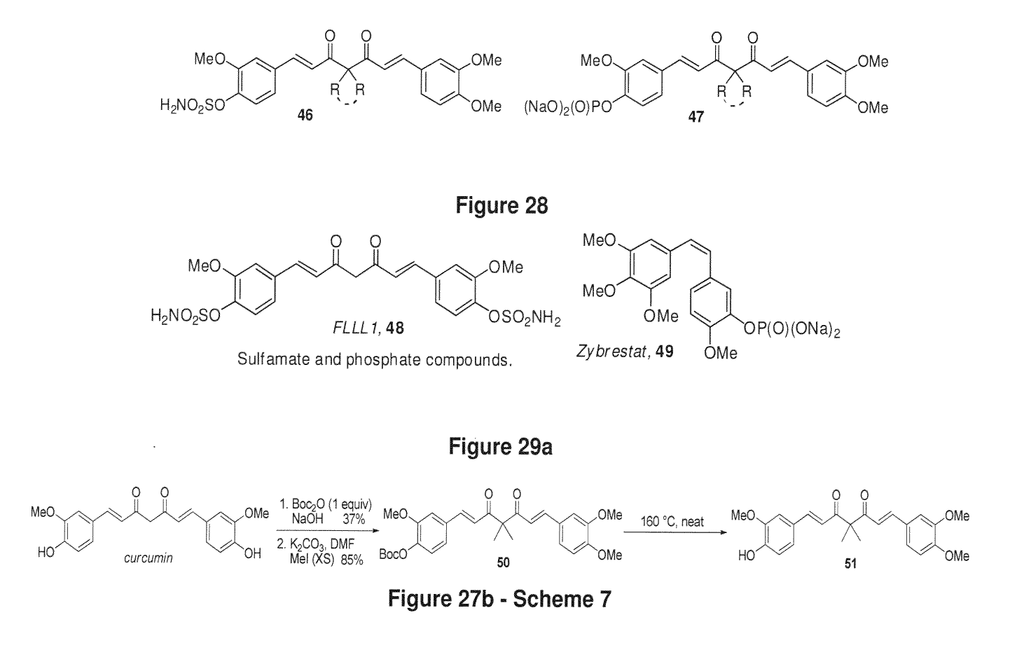

Compounds 36-38 containing the iso-butyl side chain can be prepared according to the representative synthetic plan illustrated in FIG. 27b—Scheme 6. Compounds 39 and 40 can be prepared to directly compare the relative activity of these compounds with 36.

A synthetic scheme for the preparation of 36 is shown in FIG. 27b—Scheme 6. Alkylation and subsequent Wittig olefination of the commercially available iso-vanillin (41) can provide methyl ketone 43.

Acylation of this ketone in analogy to FIG. 24c—Scheme 4 (Example 1.1.3) and alkylation of the central carbon can provide 36.

Compounds 37-40 can be prepared. For example, the preparation of 37 will necessitate the use of a Boc protected acid chloride for the acylation step and a subsequent BOC deprotection as discussed previously. Stepwise condensation of the benzaldehydes with 2,4-pentanedione can be used as an alternative strategy for the preparation of these molecules (Example 1.1.3). Successful structural modifications executed in Example 1.1 can be incorporated into the design of these non-symmetric analogues.

1.3. Pharmacological Properties of Curcumin for JAK/STAT Inhibition.

QikProp (Schrodinger LLC) was used to compute the ADME/Tox properties of FLLL31 and FLLL32 together with tamoxifen, letrozole, gemcitabine and doxorubicin. Fifty “drug-likeness” parameters have been evaluated for each compound. Strikingly, both FLLL31 and FLLL32 compounds show highly “druglike” properties.

Selected highlights for FLLL31 and 32 include: 1) metabolic stability similar to tamoxifen and gemcitabine; 2) polarities similar to letrozole and gemcitabine; 3) composite logP values similar to tamoxifen and doxorubicin; 4) the predicted IC₅₀ values for HERG K⁺ channels are close to that of letrozole, better than tamoxifen; 5) the predicted Caco-2 and MCDK cell permeability values are excellent (over 1,000); 6) the predicted brain/blood partition coefficients are between −1.8 to −0.8, which is excellent; 7) the predicted index of binding to human serum albumin ranges from 0.4 to 0.8, well within recommended range of −1.5-1.5; 8) the predicted human oral absorption percentage ranges from 97% to 100%. For overall index, FLLL32 is 80% similar to Cilnidipine, Pirozadil, Mibefradil, Binifibrate and Clobenoside. Overall, this shows that the dialkylated curcumin analogues can have reasonable pharmacological properties.

1.3.1 Structural Analogue—Sulfamates and Phosphates

Two classes of structural analogues, sulfamates and phosphates (FIG. 28), can also be synthesized in order to improve upon the oral bioavailability and water solubility of the compounds, respectively. Furthermore, these structural modifications are now believed by the inventors herein to be useful, in certain embodiments, to improve the stability of the phenolic moieties. The sulfamate derivatives of various steroids including estradiol have shown increased absorption, leading to increased activity.

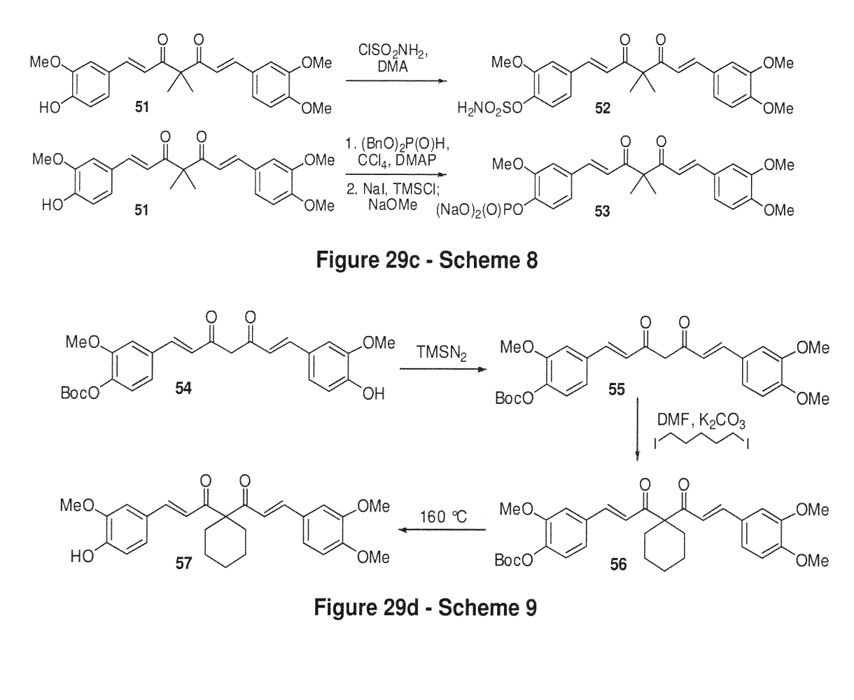

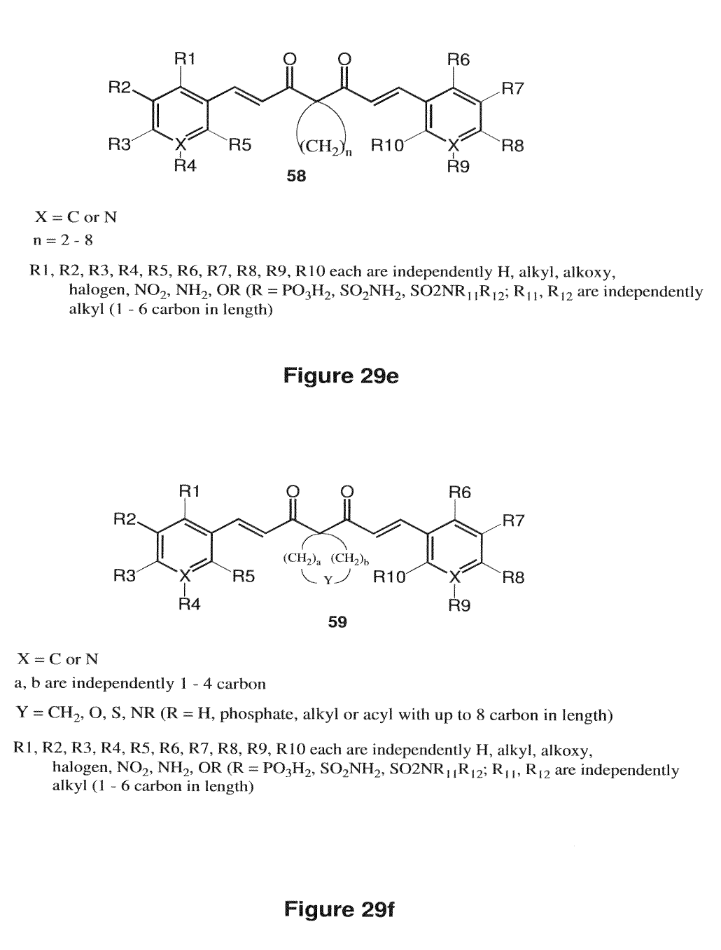

The bis-sulfamate derivative of curcumin FLLL1 (48, FIG. 29) shows a four-fold increase in potency against MCF-7 cells compared to curcumin itself. The phosphate derivative has been chosen from among other possible water-solubilizing groups based upon reported success in similar phenolic compounds. For example, the phosphate disodium salt of combretastatin, Zybrestat (49), has overcome significant solubility and stability issues to progress to phase III clinical trials as an anticancer agent.

Compound 51 is useful as a model for the synthesis of these compounds. The synthesis of 51 has been achieved utilizing a three step procedure. Mono-protection of one phenolic oxygen of curcumin as the Boc derivative was accomplished in modest yield. Treatment of this product with excess iodomethane in the presence of potassium carbonate resulted in alkylation of the remaining phenol, as well as the dialkylation of the central carbon, to give 50 in 85% yield. Finally, deprotection of the phenol via thermolytic removal of the Boc group provided 51. Further conversion of 51 to the corresponding sulfamate derivative 52 can be accomplished upon treatment with sulfamoyl chloride (FIG. 29c—Scheme 8, eq. 1).

The conversion of 51 to the disodium phosphate derivative 53 can be carried out according to the procedure of Pettit (FIG. 33c—Scheme 8, eq. 2). In this case, treatment with dibenzyl phosphite, followed by removal of the benzyl protecting groups using sodium iodide and chlorotrimethylsilane can provide the water soluble analogue. If attempts to remove the benzyl protecting groups fail, alternative procedures utilized by Pettit to install and deprotect the analogous t-butyl and trimethylsilly ethyl (SEM) derivatives can be used.

1.3.2 Cyclohexyl-Containing Derivatives

The synthesis of the corresponding cyclohexyl-containing derivatives can be carried out as illustrated in FIG. 29d—Scheme 9.

The Boc protected curcumin 54 can be methylated selectively on the phenolic oxygen. The diazomethane is useful for the conversion of curcumin to dimethoxycurcumin. In this example, the safer trimethylsilyl-diazomethane can be employed in order to carry out the methylation. An alternative strategy employs dimethylsufate and potassium carbonate in benzene to effect the same transformation. With 55 in hand, the alkylation to provide the cyclohexane ring and the subsequent deprotection can be carried out. The conversion of 57 to the sulfamate and phosphate derivatives can be accomplished in analogy to the conversion of the dimethyl compounds in FIG. 29—Scheme 8.

1.3.3 Additional Structural Analogues

FIG. 29e shows examples of analogues for compound 58. FIG. 29f shows examples of analogues for compound 59.

1.4. Validation of the JAK and STAT Binding Models.

The physical interaction of the novel small molecules with the JAK2 catalytic domain and STAT3 SH2 domain was evaluated.

1.4.1. SH2 Domain Purification.

Full-length murine STAT3 was cloned by RT-PCR in pcDNA 3.1 CTGFP TOPTO as instructed by the manufacturer (Invitrogen) and used to transform E. coli DH5. To produce a glutathione-S-transferase (GST) fusion of the SH2 domain (Y575-C687), the full-length plasmid was used as a PCR template with forward primer 5-GTAC-GGATCC-TAT ATC TTG GCC CTT TGG AA [SEQ ID NO:1] and reverse primer 5-GTCA-CTCGAG-CAG TAC TTT CCA AAT GCC TC, [SEQ ID NO:2] containing BamH1 and Xho1 restriction sites, respectively.

The PCR product and pGEX 4T-3 vector (Pharmacia) were digested with BamH1 and Xho1, and ligated to fuse the SH2 domain C-terminal to GST. E. coli BL21 were then transformed with a GST-STAT3-SH2 or empty pGEX 4T-3 plasmid and induced with IPTG. Bacteria were sonicated in PBS, extracted with 1% Triton x-100 and the protein was purified on GSH-sepharose, and western blotted for GST. The inventors have made GST-STAT3-SH2 (Y575-C687) fusion protein (MW 40 kDa) at the expected molecular weights (Western not shown).

1.4.2. Jak2 Catalytic Domain Purification.

The purification can be carried out by published procedures. The kinase domain of human JAK2 (residues 835-1132) can be cloned into pFastBac, which allows the protein to be expressed fused to a GST cleavable tag. Recombinant bacmid DNA containing the JAK2 insert can be isolated and transfected to Sf9 insect cells. Baculovirus obtained from the transfection can be used to infect Sf9 cells grown in suspension to a density of 2×10⁶ cells per mL at a multiplicity of infection greater than 10 and harvested 48 hours after infection. Cells can be resuspended in a buffer consisting of 20 mM Tris HCl, pH 8.5, 250 mM NaCl, 0.5% thesit, 5% glycerol, and 1 mM DTT supplemented with complete protease inhibitors mixture (Roche Diagnostics, Mannheim, Germany), lysed by sonication, and centrifuged at 45 000 g for 1 hour. The supernatant can be filtered and recirculated onto a GST resin (Scientifix, Victoria, Australia). After extensive washes, the fusion protein can be eluted, and fractions containing GST-JAK2 pooled and concentrated to 2 mL and incubated with α-thrombin (Sigma, St Louis, Mo.) overnight at 4° C. The protein can then be loaded onto Superdex 75 gel filtration column (HiLoad 16/60) equilibrated in 20 mM Tris pH 8.5, 250 mM NaCl, and 1 mM DTT. JAK2 can be pooled and concentrated to 10 mg/mL for crystallization trials. Crystals can then be grown at 20° C. using the hanging drop vapor-diffusion method with a reservoir solution containing 28% polyethylene glycol 4000, 0.2 M ammonium acetate, and 0.1 M citrate pH 6.0.

1.4.3. Crystal Structures of Jak2/Inhibitor and SH2/Inhibitor Complexes.

Purified JAK2 and STAT3 SH2 proteins can be crystallized through either focused screening conditions that have yielded crystals published in the literature or sparse matrix screenings. Inhibitors can be either soaked into native crystals or cocrystallized with native proteins. The structure can be solved through molecular replacement with Jak2 or SH2 apo structure as probe.

Interaction with Other Example.

As shown in FIG. 30, analogues synthesized in Example II can be tested, as described in Example III herein for improved potency, JAK2/STAT3 specificity of inhibition, cell kill, and anti-angiogenic activity and those compounds found active in cells culture can be taken on to the human tumor xenograft models. Further, compounds synthesized in Example II can be tested for improved PD and PK parameters, as described in Example IV herein.

### Example III

The inventors herein have now shown that the parental compound FLLL32 reduces blood vessel density of MDA-MB-231 STAT3 over-expressing breast cancer cells implanted into the CAM (FIG. 15b). Thus, while not wishing to be bound by theory, the inventors herein now believe that the selective FLLL32 analogues will have significant anti-angiogenic activity in angiogenesis mediated via VEGF.

Chorioallantoic Membrane (CAM) Assay of VEGF-Mediated Angiogenesis.

The CAM assay is a standard assay for testing anti-angiogenic agents. In this assay, purified VEGF is added locally to the highly vascularized CAM to induce angiogenesis. Inhibitors are then added to the same localized area of the membrane and after an incubation period, the blood vessel density of treated area of the CAM counted. The CAM assay used in these studies was modified. In this assay fertile leghorn chicken eggs are allowed to grow until 10 days of incubation, a time when most vasculogenesis has stopped and blood vessel formation is mostly through angiogenesis. The pro-angiogenic factor human VEGF-165 (100 ng) is then added to saturation to a 3 mm microbial testing disk and placed onto the CAM by breaking a small hole in the superior surface of the egg. Anti-angiogenic compounds are added 8 hr after the VEGF/bFGF at saturation to the same microbial testing disk and embryos allowed to incubate an additional 40 hr. After 48 hr, the CAMs are perfused with 4% paraformaldehyde with 0.05% Triton X-100, excised around the area of treatment, fixed again in 4% paraformaldehyde, placed onto Petri dishes in paraformaldehyde, and a digitized image taken using a dissecting microscope and CCD imaging system (Retiga, Burnaby, BC). A 1×1-cm grid is then added to the digital CAM images and the average number of vessels within 5-7 grids counted as a measure of vascularity. SU5416 is used as a positive control for anti-angiogenic activity. Data are graphed as a percent of CAMs receiving bFGF/VEGF and IC50 values estimated from 2-3 separate experiments (n=5-11) using sigmoidal dose-response relation analysis with Prism 3.0 software (GraphPad).

CAM Xenograft Assay.

In this variation of the CAM assay above, 10 DI chicken embryos are implanted with 250,000 MDA-MB-435 metastatic breast cancer cells just under the CAM in a relatively avascular area (i.e., away from large vessels). Compounds are then pipetted directly onto the CAM into the systemic circulation on a mg/kg basis one day after tumor implantation. Three days after implantation, CAMs are fixed as in the original CAM assay above, excised around the tumors, and imaged as described in the original CAM assay above. Further, fixed CAMs can be immunohistochemically stained against the angiogenic markers VEGF, MMP-2 and MMP-9.

Again, while not wishing to be bound by theory, the inventors herein now believe that the new analogues of FLLL32 will exhibit potent and selective activity in breast cancer cell lines with elevated levels of STAT3 phosphorylation.

### Example IV

### Pharmacokinetic, Metabolism, and Dose Finding Studies of Curcumin Analogue Inhibitors

Background: Curcumin Pharmacokinetics and Metabolism

Curcumin undergoes reduction of the alkyl chains and glucuronidation and sulfation of the aromatic hydroxyl groups. This contributes to the low bioavailability of curcumin when administered through any route. Oral administration is especially low due both to metabolism and poor intestinal absorption of the parent compound. Studies with radiolabeled curcumin indicated approximately 60% of the oral dose is absorbed in rats, and this percentage remained constant between 10 and 400 mg/kg doses. The majority of absorbed material is metabolized in the intestinal wall resulting in low systemic exposure of curcumin. Likewise, glucuronidated and sulfated metabolites, but not parent compound, can be found in urine.

Pharmacokinetics, Metabolism, and Dose Finding Studies of Novel Inhibitors.

Curcumin is metabolized rapidly thus limiting its in vivo exposure. With the FLLL32 analogues described in Example II maintain the keto form and prevent tautomerization to the enol form, as well as methylation of the aromatic hydroxyl groups, the primary pathways for metabolic transformation of curcumin (glucuronidation, sulfation and reduction will be hindered or blocked completely.

Therefore, the inventors now believe that there will be improved bioavailability and tissue absorption of the FLLL32 analogues, thus enabling achievement of therapeutic concentrations in vivo. This improved disposition will increase activity and efficacy compared to other JAK2/STAT3 inhibitors and curcumin.

For each compound, detailed PK data can be generated, including information on bioavailability through oral and IP routes of administration, dose dependence of PK, in vivo distribution and metabolism (including in vitro metabolism), and overall disposition of each inhibitor. Collectively, this data enables modeling and rational design of optimized dosing regimens for efficacy determination in tumor-bearing mice.

It is to be noted that the inventors' approach greatly differs from the typical approaches whereby maximally-tolerated doses are utilized in long-term efficacy studies. These approaches are often applied without prior knowledge of PK, thus increasing the chance for selection of a sub-optimal dosing schedule or continued development and evaluation of a compound with poor PK properties. Additionally, multiple compounds are often compared using a single dosing regimen. If PK differs among these compounds, the comparisons may lead to incorrect conclusions. In contrast, the inventors' approach fully characterizes PK relationships and insures maximal and tolerable exposure is achieved for efficacy determination of each compound.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

The publication and other material used herein to illuminate the invention or provide additional details respecting the practice of the invention, are incorporated be reference herein, and for convenience are provided in the following bibliography.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

1. (canceled)

2. A curcumin analogue comprising 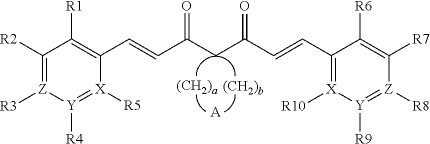
X, Y, Z═C or N
a, b are independently 1-4 carbon
A=CH₂, O, S, NR (R═H, phosphate, alkyl or acyl with up to 8 carbon in length)
R1, R2, R3, R4, R5, R6, R7, R8, R9, R10 each one independently H, alkyl, alkoxy, halogen, NO₂, NH₂, OR, (R=PO₂H₂, SO₂NH₂, SO2NR₁₁R₁₂; R₁₁, R₁₂ are independently alkyl (1-6 carbon in length)

3. Curcumin analogue of claim 2, having the formula 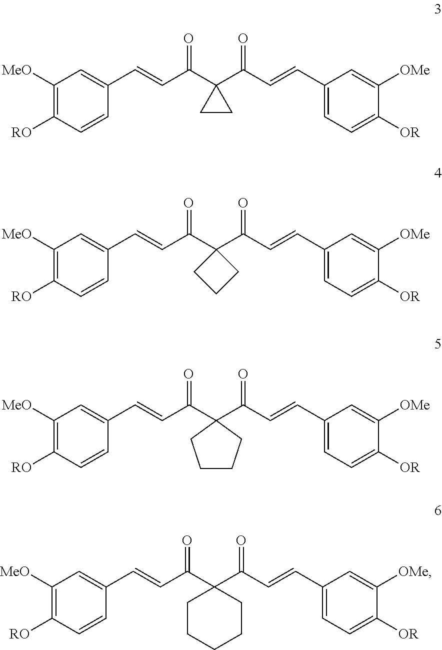
wherein R=Me.

4. (canceled)

5. (canceled)

6. (canceled)

7. A method for making a curcumin analogue comprising synthesizing using Scheme 1

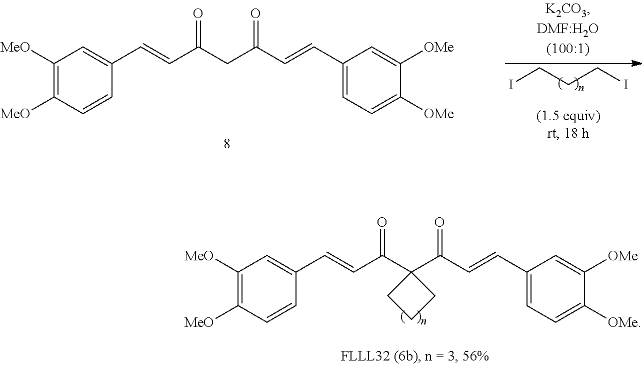

8. (canceled)

9. (canceled)

10. (canceled)

11. (canceled)

12. (canceled)

13. Curcumin analogues of claim 2, having a benzaldehyde aromatic substituent selected from one or more of: 21, 22, 23, as shown in FIG. 21

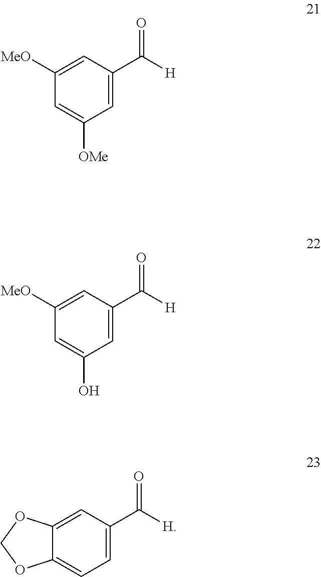

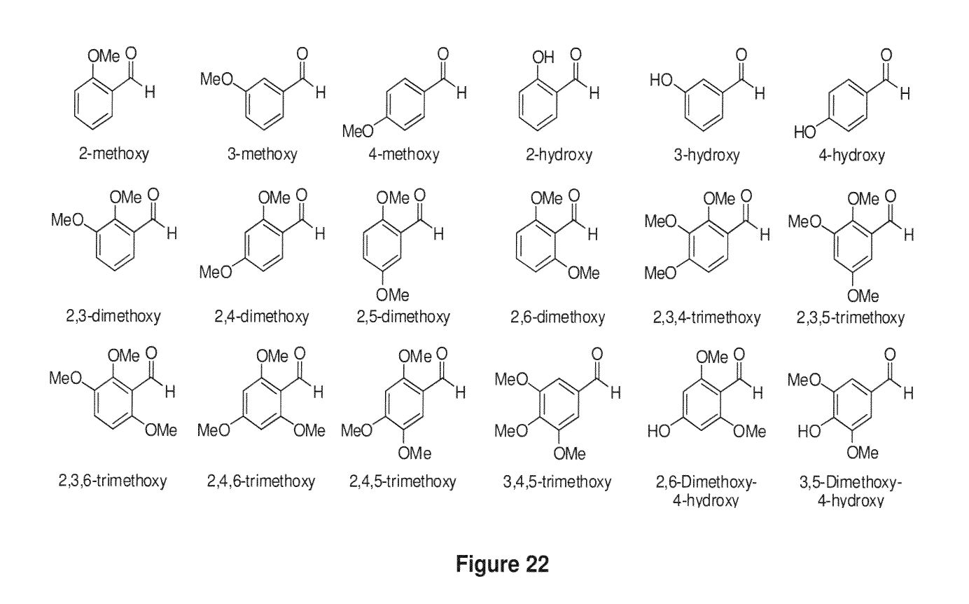

14. Curcumin analogues of claim 2, having a having a mono-, di-, and tri-substituted benzaldehyde substituent containing only methoxy (and hydroxy) group, as shown in FIG. 22

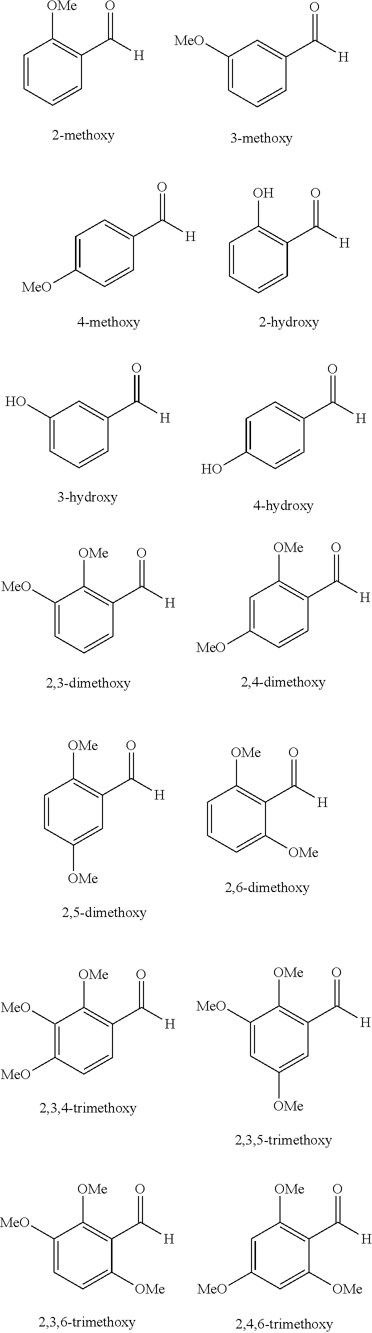

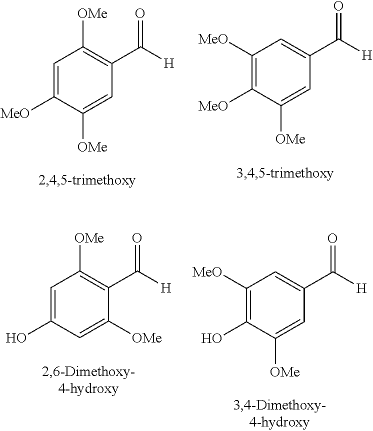

15. Curcumin analogues of claim 2, comprising one or more of 24 and 25, as shown in FIG. 23a

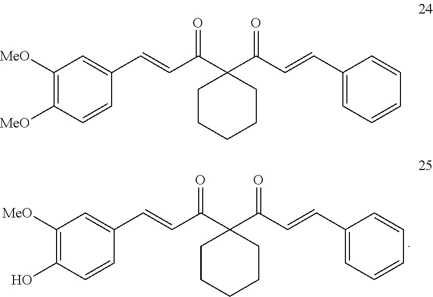

16. (canceled)

17. A method for making a curcumin analogue comprising synthesizing using Scheme 3, as shown in FIG. 23b

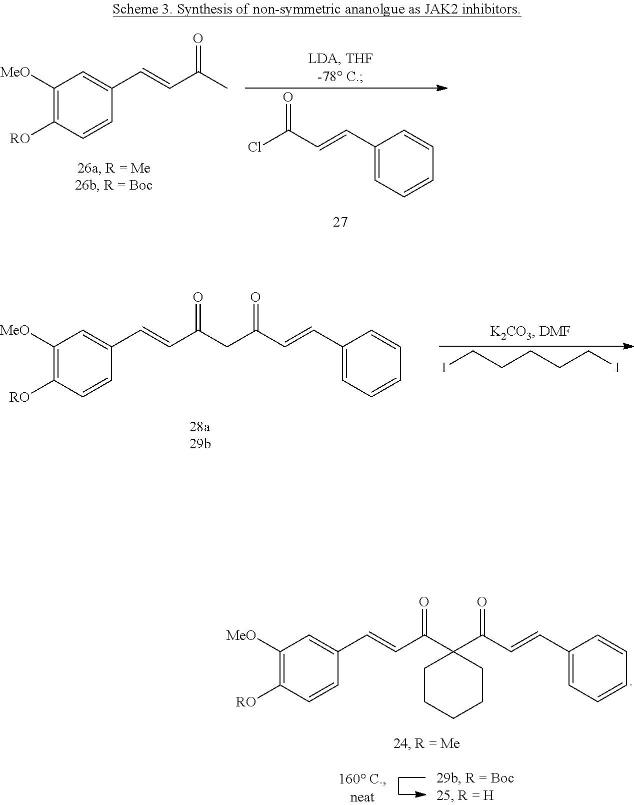

18. (canceled)

19. (canceled)

20. Curcumin analogues of claim 2, comprising cyclohexyl derivatives 6a, as shown in FIG. 25a

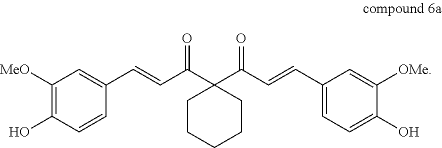

21. Curcumin analogues of claim 2, comprising one or more of: 6b, 5b, 32, 33, 34, 35, as shown in FIG. 26a

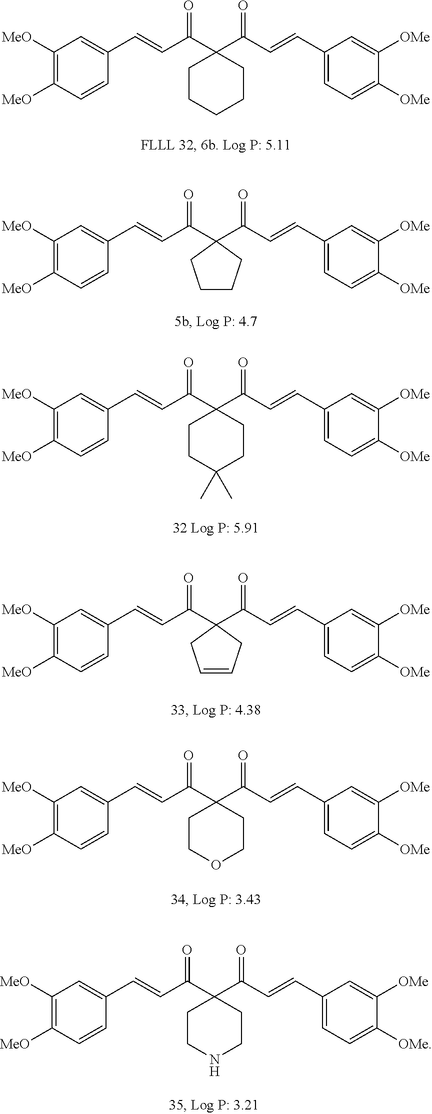

22. (canceled)

23. A method for making a curcumin analogue of claim 2, comprising synthesizing using Scheme 5, as shown in FIG. 26b

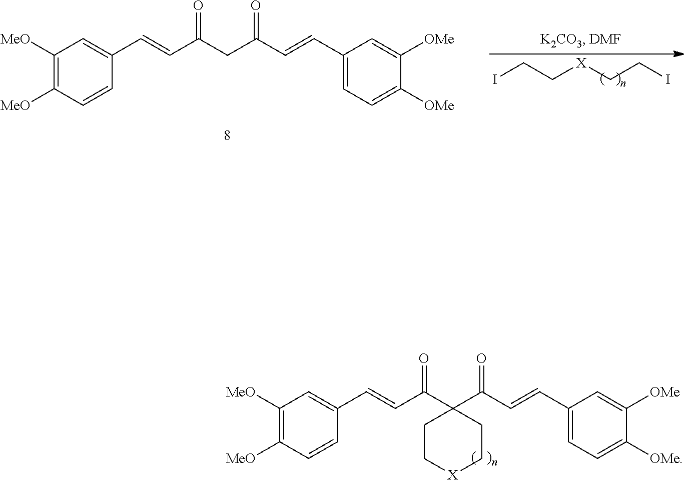

24. Curcumin analogues of claim 2, comprising one or more of: 36, 37, 38, 39, 40, as shown in FIG. 27a

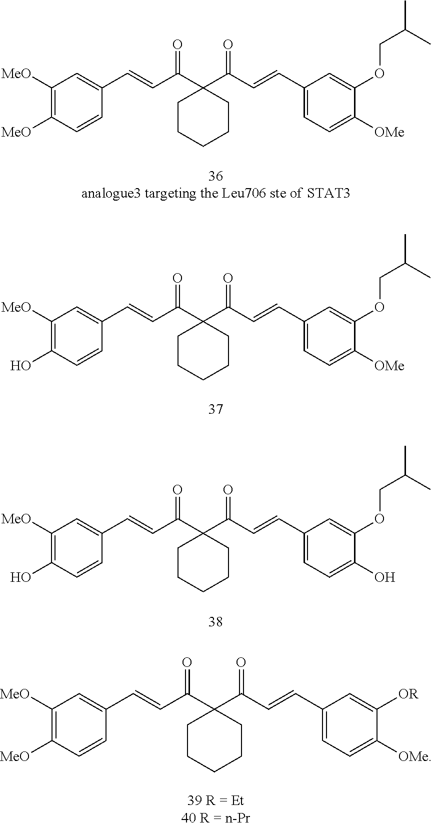

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A pharmaceutical composition at least one curcumin analogue of claim 2.

39. A method of treating a cancer-related disease comprising modulating the activity of a one or more of JAK and STAT in a subject in need thereof, by administering at least one curcumin analogue of claim 2.

40. A method for inhibiting JAK/STAT signaling in a subject in need thereof, comprising administering one or more of the curcumin analogues claim 2.

41. A chemotherapeutic composition comprising one or more of the curcumin analogues of claim 2.

42. A chemopreventive composition comprising one or more of the curcumin analogues of claim 2.

43. A composition comprising an effective amount of at least one curcumin compound of claim 2, wherein the compound in the effective amount is capable of inhibiting STAT3 phosphorylation in a cell.

44. A method for treating cancer, or for preventing the incidence or the recurrence of cancer in an subject, comprising:
administering to the subject an effective amount of at least one curcumin analogue compound of claim 2,
wherein the compound in the effective amount is capable of inhibiting the STAT3 phosphorylation signaling pathway in certain cells of the subject.

45. A method for inhibiting STAT3 phosphorylation in an subject with aberrant STAT3 signaling, comprising:
i) determining whether the signaling is abnormal in the subject; and
ii) administering to the subject a STAT3 phosphorylation inhibiting curcumin compound of claim 2.

46. A method of screening for a potential therapeutic agent for treating or preventing cancer, the method comprising the steps of:
i) contacting a cell with an intact STAT3 signaling pathway with a candidate molecule;
ii) monitoring changes in the activation of the STAT3 signaling pathway;
iii) determining whether the molecule is capable of inhibiting the STAT3 signaling pathway; and
iv) identifying the molecule as a potential therapeutic agent if it is determined to be capable of inhibiting the STAT3 signaling pathway.

47. A pharmaceutical composition comprising a curcumin analog conjugate according to claim 2, and at least one pharmaceutically acceptable carrier.

48. A method of treatment or prevention of a disease selected from cancer, diabetes, and inflammatory diseases in a subject in need thereof, the method comprising:
administering a therapeutically effective amount of a curcumin analog conjugate according to claim 2 to the subject.

49. The method of claim 48, wherein the disease is a cancer.

50. The method of claim 48, wherein the disease is a breast cancer.

51. The method of claim 48, wherein the disease is a pancreatic cancer.

52. (canceled)
